(19) United States
(12) Patent Application Publication
(10) Pub. No.: US 2007/0082892 A1
(43) Pub. Date: Apr. 12, 2007

(54) Benzotriazepine derivatives and their use as gastrin and cholecystokinin receptor ligands

(75) Inventors: John Spencer, London (GB); Iain Mair McDonald, Lomdon (GB); Ian Duncan Linney, London (GB)

Correspondence Address: PROSKAUER ROSE LLP, 1001 PENNSYLVANIA AVE, N.W.,, SUITE 400 SOUTH, WASHINGTON, DC, 20004, US

(73) Assignee: James Black Foundation Limited, London (GB)

(21) Appl. No.: 10/556,576

(22) PCT Filed: May 12, 2004
(86) PCT No.: PCT/GB04/02049

§ 371 Date: Jan. 3, 2007

(30) Foreign Application Priority Data
May 12, 2003 (GB) 03108644

(51) Int. Cl. A61K 31/5513; C07D 403/02; C07D 255/04

(52) U.S. Cl. 514/221; 540/501

(57) ABSTRACT

This invention relates to a compound of formula (I). The compound is useful for the treatment of gastrin related disorders.

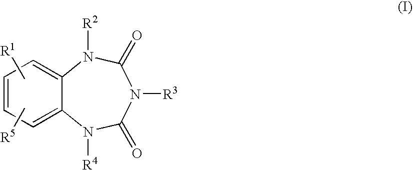

This application claims priority from U.K. patent application No. 0310864.4, the entire contents of which is hereby incorporated by reference.

This invention relates to gastrin and cholecystokinin (CCK) receptor ligands. (The receptor previously known as the CCK_{B}/gastrin receptor is now termed the CCK₂ receptor). The invention also relates to methods for preparing such ligands and to compounds which are useful intermediates in such methods. The invention further relates to pharmaceutical compositions comprising such ligands and methods for preparing such pharmaceutical compositions.

Gastrin and the cholecystokinins are structurally related neuropeptides which exist in gastrointestinal tissue and the central nervous system (Mutt V., Gastrointestinal Hormones, Glass G. B. J., ed., Raven Press, New York, p. 169; Nisson G., ibid., p. 127).

Gastrin is one of the three primary stimulants of gastric acid secretion. Several forms of gastrin are found including 34-, 17- and 14-amino acid species with the minimum active fragment being the C-terminal tetrapeptide (TrpMetAspPhe-NH₂) which is reported in the literature to have full pharmacological activity (Tracy H. J. and Gregory R. A., Nature (London), 1964, 204, 935). Much effort has been devoted to the synthesis of analogues of this tetrapeptide (and the N-protected derivative Boc-TrpMetAspPhe-NH₂) in an attempt to elucidate the relationship between structure and activity.

Natural cholecystokinin is a 33 amino acid peptide (CCK-33), the C-terminal 5 amino acids of which are identical to those of gastrin. Also found naturally is the C-terminal octapeptide (CCK-8) of CCK-33.

The cholecystokinins are reported to be important in the regulation of appetite. They stimulate intestinal mobility, gall bladder contraction, pancreatic enzyme secretion and are known to have a trophic action on the pancreas. They also inhibit gastric emptying and have various effects in the central nervous system.

Compounds which bind to cholecystokinin and/or gastrin receptors are important because of their potential pharmaceutical use as antagonists, inverse agonists or partial agonists of the natural peptides. Such compounds are described herein as ligands. The term ligand as used herein means either an antagonist, partial or full agonist, or an inverse agonist. Usually, the term ligand refers to an antagonist.

A number of gastrin ligands have been proposed for various therapeutic applications, including the prevention of gastrin-related disorders including gastrointestinal ulcers, dyspepsia, reflux oesophagitis (gastroesophageal reflux disease (GERD), both erosive and non-erosive) by reduction in gastric acid secretion and/or improving impaired motor activity at the lower oesophageal sphincter, Zollinger-Ellison syndrome, Barrett's oesophagus (specialized intestinal metaplasia of distal oesophagus), ECL cell hyperplasia, rebound hypersecretion (following cessation of anti-secretory therapy), ECL-derived gastric polyps most commonly found in patients with atrophic gastritis both with (pernicious anaemia) or without vitamin B12 deficiency, antral G cell hyperplasia and other conditions in which lower gastrin activity or lower acid secretion is desirable. The hormone has also been shown to have a trophic action on cells and so an antagonist may be expected to be useful in the treatment of cancers, particularly in the GI tract, more particularly in the stomach, oesophagus and colo-rectal areas. Tumours found in other organs such as the pancreas, lung (small cell lung carcinomas) and thyroid (thyroid medullary tumours) may also be treated.

Other possible uses are in the potentiation of opiate (for example morphine) analgesia. Moreover, ligands for cholecystokinin receptors in the brain (so-called CCK₂ receptors) have been claimed to possess anxiolytic activity.

1,5-benzodiazepine-2,4-diones are established Gastrin/CCK₂ antagonists (see, for example, Ursini et al., J. Med. Chem., (2000), 43(20), 3596-3613; Finzia et al., Bioorg. Med Chem. Letts., (1996), 6(24), 2957-2962; Curotto, et al., Bioorg. Med. Chem. Letts., (1995) 5(24), 3011-3016; Bailey et al., Bioorg. Med. Chem. Letts., (1995) 7(3), 281-286 and Hagishita, et al., Bioorg. Med. Chem., (1997), 5 (7), 1433-1446).

However, for optimal activity and selectivity, 1,5-benzodiazepine-2,4-diones are often resolved into their (S)-form. An example of this is in compound A below, (see, for example, Bioorg. Med. Chem. Lett. 1996, 5, 2957-2962 and ibid. 1997, 7, 281-286 and WO9425445). The requirement for a single enantiomer of the 1,5-benzodiazepine-2,4-dione gastrin ligands is undesirable. The synthesis of single enantiomers from achiral precursors, as in these cases, is a costly and relatively complex procedure. This generally requires, for example, either a separation step, usually inefficient as one enantiomer is discarded, or the use of an often expensive chiral auxiliary during the synthesis, coupled with an increase in chemical steps. For these reasons, a drug candidate with no stereocentres on the seven-membered ring would offer a distinct advantage over chiral alternatives. One approach that avoids the need for resolution was described in Bioorg. Med. Chem., 1997, 5, 1433-1446, whereby the 1,5-benzodiazepine-2,4-dione unit contains identical groups on the 1- and 5-N positions, leading to an internal plane of symmetry as in compound B below. However, this approach obviously limits the diversity of the groups on the seven-membered ring.

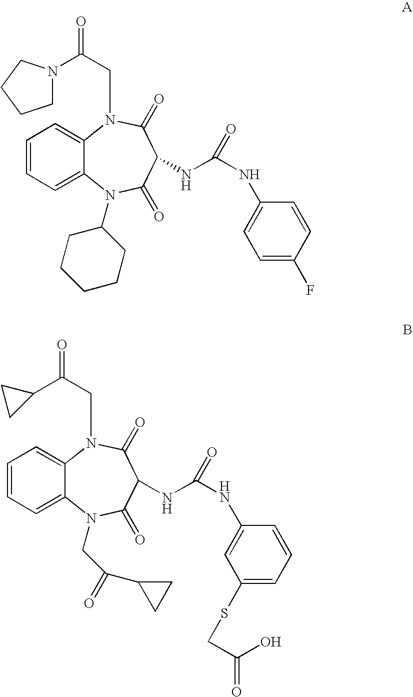

It is an object of the present invention to provide potent and selective gastrin and CCK receptor ligands. It is a further object of the present invention to provide gastrin and CCK receptor ligands which have no chiral centre on the 7-membered ring and which can therefore be prepared using straightforward synthetic methods. It is a further object of the present invention to provide gastrin and CCK receptor ligands that can have different groups on the 1- and 5-N atoms.

Simple 1,3,5-benzotriazepine-2,4-diones have been described in Weigert et al., Journal of Organic Chemistry (1973), 38, 1316; Clifford et al., Pesticide Science (1976), 7(5), 453-458 and in German patent publication number DE2036172 (1972): Chem. Abs., 76, 127031. However, these compounds do not fall within the scope of the present invention.

According to the present invention, there are provided compounds of formula (I): 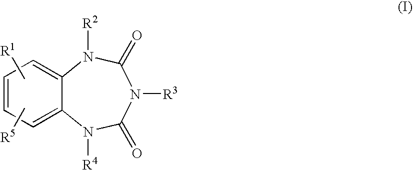
wherein:

R¹ and R⁵ are independently H, C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino, (C₁ to C₆ alkyl)aminocarbonyl, di(C₁ to C₆ alkyl)aminocarbonyl, [N-Z](C₁ to C₆ alkyl)carbonylamino, formyloxy, formamido, (C₁ to C₆ alkyl)aminosulfonyl, di(C₁ to C₆ alkyl)aminosulfonyl, [N-Z](C₁ to C₆ alkyl)sulfonylamino or cyano; or R¹ and R⁵ together form a methylenedioxy group;

R² is an optionally substituted C₁ to C₁₈ hydrocarbyl group wherein up to three C atoms may optionally be replaced by N, O and/or S atoms;

R³ is —(CR¹¹R¹²)ₘ—X—(CR¹³R¹⁴)ₚ-R⁹;

m is 0, 1, 2, 3 or 4 preferably 1 or 2);

p is 0, 1 or 2;

X is a bond, —CR¹⁵═CR¹⁶—, —C≡C—, C(O)NH, NHC(O), C(O)NMe, NMeC(O), C(O)O, NHC(O)NH, NHC(O)O, OC(O)NH, NH, O, CO, SO₂, SO₂NH, C(O)NHNH,

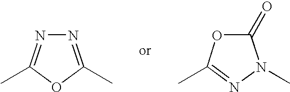

R⁹ is H; C₁ to C₆ alkyl; or phenyl, naphthyl, pyridyl, benzimidazolyl, indazolyl, quinolinyl, isoquinolinyl, tetrahydroisoquinolinyl, indolinyl, isoindolinyl, indolyl, isoindolyl or 2-pyridonyl, all optionally substituted with 1, 2 or 3 groups independently selected from -L-Q

wherein:

L is a bond, or a group of the formula —(CR¹⁷R¹⁸)ᵥ—Y—(CR¹⁷R¹⁸)_{w}, wherein v and w are independently 0, 1, 2 or 3, and Y is a bond, —CR¹⁵═CR¹⁶—, phenyl, furanyl, thiophenyl, pyrrolyl, thiazolyl, imidazolyl, oxazolyl, isoxazolyl, pyrazolyl, isoxazolonyl, piperazinyl, piperidinyl, morpholinyl, pyrrolidinyl, isothiazolyl, triazolyl, oxadiazolyl, thiadiazolyl, pyridyl or pyridazyl; and

Q is H, (C₁ to C₆ alkyl)oxy, [N-Z](C₁ to C₆ alkyl)oxy(C₁ to C₆ alkyl)amino, thio, (C₁ to C₆ alkyl)thio, carboxy(C₁ to C₆ alkyl)thio, carboxy, carboxy(C₁ to C₆ alkyl), carboxy(C₁ to C₆ alkenyl), [N-Z]carboxy(C₁ to C₆ alkyl)amino, carboxy(C₁ to C₆ alkyl)oxy, formyl, (C₁ to C₆ alkyl)carbonyl, (C₁ to C₆ alkyl)oxycarbonyl, (C₁ to C₆ alkyl)oxycarbonyl(C₁ to C₆ alkyl)thio, (C₁ to C₆ alkyl)carbonyloxy, nitro, trihalomethyl, hydroxy, amino, [N-Z](C₁ to C₆ alkyl)amino, aminocarbonyl, (C₁ to C₆ alkyl)aminocarbonyl, di(C₁ to C₆ alkyl)aminocarbonyl, [N-Z](C₁ to C₆ alkyl)carbonylamino, C₅ to C₈ cycloalkyl, [N-Z](C₁ to C₆ alkyl)carbonyl(C₁ to C₆ alkyl)amino, halo, halo(C₁ to C₆ alkyl), sulfamoyl, [N-Z](C₁ to C₆ alkyl)sulfonylamino, (C₁ to C₆ alkyl)sulfonylaminocarbonyl, carboxy(C₁ to C₆ alkyl)sulfonyl, carboxy(C₁ to C₆ alkyl)sulfinyl, tetrazolyl, [N-Z]tetrazolylamino, cyano, amidino, amidinothio, SO₃H, formyloxy, formamido, C₃ to C₈ cycloalkyl, (C₁ to C₆ alkyl)sulphamoyl, di(C₁ to C₆ alkyl)sulphamoyl, (C₁ to C₆ alkyl)carbonylaminosulfonyl, 5-oxo-2,5-dihydro[1,2,4]oxadiazolyl, carboxy(C₁ to C₆ alkyl)carbonylamino, tetrazolyl(C₁ to C₆ alkyl)thio, [N-Z]tetrazolyl(C₁ to C₆ alkyl)amino, 5-oxo-2,5-dihydro[1,2,4]thiadiazolyl, 5-oxo-1,2-dihydro[1,2,4]triazolyl, [N-Z](C₁ to C₆ alkyl)amino(C₁ to C₆ alkyl)amino, a group of the formula 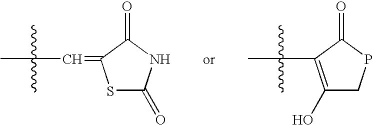
wherein P is O, S or NR¹⁹, or benzyloxycarbonyl(C₁ to C₆ alkyl)thio comprising a phenyl group that is optionally substituted with 1, 2 or 3 groups independently selected from C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano;

Z is H, C₁ to C₆ alkyl, t-butoxycarbonyl, acetyl, benzoyl or benzyl;

R⁴ is an optionally substituted C₁ to C₁₈ hydrocarbyl group wherein up to three C atoms may optionally be replaced by N, O and/or S atoms;

R¹¹, R¹², R¹³, R¹⁴, R¹⁵, R¹⁷, R¹⁸ and R¹⁹ are independently H or C₁ to C₃ alkyl; and

R¹⁶ is H, C₁ to C₃ alkyl, or acetylamino;

or a pharmaceutically acceptable salt thereof;

with the proviso that when R¹, R³ and R⁵ are all H and R⁴ is methyl, R² may not be methyl.

Preferably R¹ and R⁵ are both H. However, it will be appreciated the benzo-fused ring system may have one or two substituents on the benzene ring as indicated hereinabove. The substituents may have subtle steric and/or electronic effects which modify the activity of the compound at the gastrin receptor. However, the presence or otherwise of certain substituents on the benzene ring is not crucial to the overall pharmacological activity of the present compounds.

Preferably, R² is of formula:
—(CH₂)ₛ—C(R⁶R⁷)ₙ—(CH₂)ₜ-R⁸
wherein:

R⁶ and R⁷ are independently selected from H, C₁ to C₆ alkyl or OH; or R⁶ and R⁷ together represent an ═O group;

n is 0 or 1;

s is 0, 1, 2 or 3;

t is 0, 1, 2 or 3; and

R⁸ is selected from H, OH, C₁ to C₁₂ alkyl, (C₁ to C₁₂ alkyl)oxy, C₃ to C₁₂ cycloalkyl, phenyl, benzyloxy, naphthyl, pyridyl, pyrrolyl, imidazolyl, pyrazolyl, pyridazinyl, pyrimidinyl, triazolyl, furanyl, thienyl, furazanyl, oxazolyl, isoxazolyl, thiazolyl, thiazinyl, indolyl, indolinyl, isoindolyl, isoindolinyl, isoquinolinyl, quinolinyl, benzofuranyl, benzothienyl, piperazinyl, piperidinyl, pyrrolidinyl, pyrrolinyl, dihydropyranyl, tetrahydropyranyl, pyranyl, tetrahydrofuranyl, morpholinyl, thiazolidinyl, thiomorpholinyl or thioxanyl (all optionally substituted with 1, 2 or 3 groups independently selected from C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano).

More preferably R² is of the formula:
—(CH₂)ₛ—C(O)—(CH₂)ₜ-R⁸
wherein:

s is 0, 1, 2 or 3;

t is 0, 1, 2 or 3;

R⁸ is selected from H, OH, C₁ to C₁₂ alkyl, (C₁ to C₁₂ alkyl)oxy, C₃ to C₁₂ cycloalkyl, phenyl, benzyloxy, naphthyl, pyridyl, pyrrolyl, imidazolyl, pyrazolyl, pyridazinyl, pyrimidinyl, triazolyl, furanyl, thienyl, furazanyl, oxazolyl, isoxazolyl, thiazolyl, thiazinyl, indolyl, indolinyl, isoindolyl, isoindolinyl, isoquinolinyl, quinolinyl, benzofuranyl, benzothienyl, piperazinyl, piperidinyl, pyrrolidinyl, pyrrolinyl, dihydropyranyl, tetrahydropyranyl, pyranyl, tetrahydrofuranyl, morpholinyl, thiazolidinyl, thiomorpholinyl or thioxanyl (all optionally substituted with 1, 2 or 3 groups independently selected from C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano).

A further preferred group of compounds according to the present invention is where R² is of formula:
—(CH₂)C(O)R⁸
wherein:

R⁸ is a branched C₃ to C₁₂ alkyl group (such as tert-butyl, sec-butyl, isopropyl, isobutyl or isovaleryl); or R⁸ is a C₃ to C₁₂ cycloalkyl (such as cyclopentyl, cyclohexyl, cycloheptyl or adamantyl) phenyl, pyridyl, pyrrolidinyl or piperidinyl group (all optionally substituted with 1, 2 or 3 C₁₋₆ alkyl groups).

Preferably, R¹¹, R¹², R¹³, R¹⁴, R¹⁵, R¹⁶, R¹⁷, R¹⁸ and R¹⁹ are all H.

In one embodiment, Q is H, (C₁ to C₆ alkyl)oxy, [N-Z](C₁ to C₆ alkyl)oxy(C₁ to C₆ alkyl)amino, thio, (C₁ to C₆ alkyl)thio, carboxy(C₁ to C₆ alkyl)thio, carboxy, carboxy(C₁ to C₆ alkyl), carboxy(C₁ to C₆ alkenyl), [N-Z]carboxy(C₁ to C₆ alkyl)amino, carboxy(C₁ to C₆ alkyl)oxy, formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, amino, [N-Z](C₁ to C₆ alkyl)amino, aminocarbonyl, (C₁ to C₆ alkyl)aminocarbonyl, di(C₁ to C₆ alkyl)aminocarbonyl, [N-Z](C₁ to C₆ alkyl)carbonylamino, C₅ to C₈ cycloalkyl, [N-Z](C₁ to C₆ alkyl)carbonyl(C₁ to C₆ alkyl)amino, halo, halo(C₁ to C₆ alkyl), sulfamoyl, [N-Z](C₁ to C₆ alkyl)sulfonylamino, (C₁ to C₆ alkyl)sulfonylaminocarbonyl, carboxy(C₁ to C₆ alkyl)sulfonyl, carboxy(C₁ to C₆ alkyl)sulfinyl, tetrazolyl, [N-Z]tetrazolylamino, cyano, amidino, amidinothio, SO₃H, formyloxy, formamido, C₃ to C₈ cycloalkyl, (C₁ to C₆ alkyl)sulphamoyl, di(C₁ to C₆ alkyl)sulphamoyl, (C₁ to C₆ alkyl)carbonylaminosulfonyl, 5-oxo-2,5-dihydro[1,2,4]oxadiazolyl, carboxy(C₁ to C₆ alkyl)carbonylamino, tetrazolyl(C₁ to C₆ alkyl)thio, [N-Z]tetrazolyl(C₁ to C₆ alkyl)amino, 5-oxo-2,5-dihydro[1,2,4]thiadiazolyl, 5-oxo-1,2-dihydro[1,2,4]triazolyl, [N-Z](C₁ to C₆ alkyl)amino(C₁ to C₆ alkyl)amino, or a group of the formula 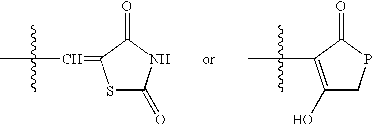
wherein P is O, S or NR¹⁹.

A preferred group of compounds according to the present invention is where R³ is of formula:
—(CH₂)—X-R⁹
wherein:

X is C(O)NH or NHC(O), more preferably X is C(O)NH.

Preferably, R⁹ is phenyl substituted with a carboxy, carboxy(C₁ to C₆ alkyl), tetrazolyl, tetrazolyl-N-(C₁ to C₆ alkyl)amino, carboxy(C₁ to C₆ alkyl)thio, (C₁ to C₆ alkyl)oxycarbonyl(C₁ to C₆ alkyl)thio, carboxy(C₁ to C₆ alkyl)sulfonyl, (C₁ to C₆ alkyl)amino, or 5-oxo-2,5-dihydro[1,2,4]oxadiazolyl group, or a benzyloxycarbonyl(C₁ to C₆ alkyl)thio group comprising a phenyl group that is optionally substituted with 1, 2 or 3 groups independently selected from C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano; or R⁹ is a N-[carboxy(C₁ to C₆ alkyl)]indolinyl or N-[carboxy(C₁ to C₆ alkyl)]indolyl group.

Equally preferably, R⁹ is phenyl substituted with a carboxy, carboxy(C₁ to C₆ alkyl), tetrazolyl, tetrazolyl-N-(C₁ to C₆ alkyl)amino, carboxy(C₁ to C₆ alkyl)thio, carboxy(C₁ to C₆ alkyl)sulfonyl, (C₁ to C₆ alkyl)amino, or 5-oxo-2,5-dihydro[1,2,4]oxadiazolyl group; or

R⁹ is a N-[carboxy(C₁ to C₆ alkyl)]indolinyl or N-[carboxy(C₁ to C₆ alkyl)]indolyl group.

When R⁹ is a substituted phenyl group, the substituent is preferably at the 3-position of the phenyl group.

Preferably, in compounds according to the present invention R⁴ is of formula:
—(CH₂)_{q}-T-R¹⁰
wherein:

q is 0, 1, 2 or 3;

T is a bond, O, S, NH or N (C₁ to C₆ alkyl); and

R¹⁰ is C₁ to C₁₂ alkyl, C₃ to C₁₂ cycloalkyl, phenyl, naphthyl, pyridyl, pyrrolyl, imidazolyl, pyrazolyl, pyridazinyl, pyrimidinyl, triazolyl, furanyl, thienyl, furazanyl, oxazolyl, isoxazolyl, thiazolyl, thiazinyl, indolyl, indolinyl, isoindolyl, isoindolinyl, isoquinolinyl, quinolinyl, benzofuranyl, benzothienyl, piperazinyl, piperidinyl, pyrrolidinyl, pyrrolinyl, dihydropyranyl, tetrahydropyranyl, pyranyl, tetrahydrofuranyl, morpholinyl, thiazolidinyl, thiomorpholinyl or thioxanyl (all optionally substituted with 1, 2 or 3 groups independently selected from C₁ to C₆ alkyl, (C₁ to C₆ alkyl)oxy, C₃ to C₈ cycloalkyl, (C₃ to C₈ cycloalkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano).

More preferably, in compounds according to the present invention, R⁴ is selected from C₁₋₁₂ alkyl (such as tert-butyl, sec-butyl, isopropyl, isobutyl or isovaleryl), C₃₋₁₂ cycloalkyl (such as cyclopentyl, cyclohexyl, cycloheptyl or adamantyl), pyridyl or phenyl (all of which may be optionally substituted with 1, 2 or 3 groups selected from OMe, NMe₂, CF₃, Me, F, Cl, Br or I).

In all compounds of the present invention, preferably q is 0 and T is a bond. More preferably R⁴ is C₃-C₁₂ cycloalkyl, and more preferably, R⁴ is cyclohexyl.

Certain compounds of the invention exist in various regioisomeric, enantiomeric, tautomeric and diastereomeric forms. It will be understood that the invention comprehends the different regioisomers, enantiomers, tautomers and diastereomers in isolation from each other as well as mixtures.

General Synthesis of 1,3,5-benzotriazepine-2,4-diones.

Simple 1,3,5-benzotriazepine-2,4-diones have been described in Weigert et al., Journal of Organic Chemistry (1973), 38, 1316; Clifford et al., Pesticide Science (1976), 7(5), 453-458 and in DE2036172. A typical route to IV is in given in Scheme 1. The condensation reaction of I to afford II has been covered in DE2036172. The alkylation of II affords the derivatives III.

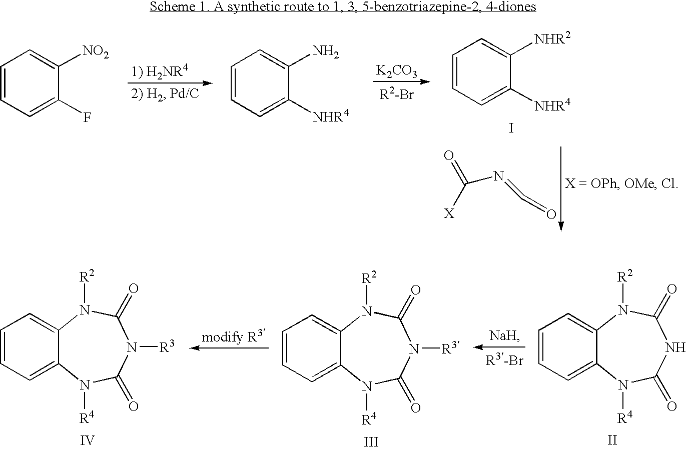

R^{3′} groups which are suitable precursors of R³ will depend on the particular nature of R³. For example, when R³ is —(CH₂)ₘC(O)NH—(CH₂)ₚ-R⁹, a suitable R^{3′} group would be —(CH₂)ₘCO₂(C₁₋₆ alkyl). In this case, the requisite R³ groups may be readily accessed via an ester hydrolysis followed by a simple amide coupling reaction. Further deprotection, if appropriate, may be necessary to reveal the final R³ group. The skilled person will be aware of many other suitable R^{3′} groups, depending on the nature of R³.

Alkylation may be performed by, for example, displacement of an alkyl halide in the presence of a base. Methods of alkylation will be readily apparent to the person skilled in the art.

Hence, the present invention also provides a method of making compounds according to formula (I).

It is an important advantage of the synthesis described hereinabove that no chiral centres are generated in the benzotriazepinone ring system during the synthesis.

The invention also comprehends derivative compounds (“pro-drugs”) which are degraded in vivo to yield the species of formula (I) Pro-drugs are usually (but not always) of lower potency at the target receptor than the species to which they are degraded. Pro-drugs are particularly useful when the desired species has chemical or physical properties which make its administration difficult or inefficient. For example, the desired species may be only poorly soluble, it may be poorly transported across the mucosal epithelium, or it may have an undesirably short plasma half-life. Further discussion of pro-drugs may be found in Stella, V. J. et al., “Prodrugs”, Drug Delivery Systems, 1985, pp. 112-176, and Drugs, 1985, 29, pp. 455-473.

Pro-drug forms of the pharmacologically-active compounds of the invention will generally be compounds according to formula (I) having an acid group which is esterified or amidated. Included in such esterified acid groups are groups of the form —COOR^{a}, wherein R^{a} is C₁ to C₅ alkyl, phenyl, substituted phenyl, benzyl, substituted benzyl, or one of the following:

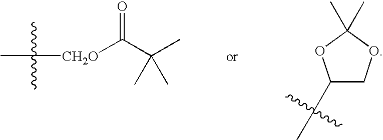

Amidated acid groups include groups of the formula —CONR^{b}R^{c}, wherein R^{b} is H, C₁ to C₅ alkyl, phenyl, substituted phenyl, benzyl, or substituted benzyl, and R^{c} is —OH or one of the groups just recited for R^{b}.

Compounds of formula (I) having an amino group may be derivatised with a ketone or an aldehyde such as formaldehyde to form a Mannich base. This will hydrolyse with first order kinetics in aqueous solution.

Another aspect of the present invention is a pharmaceutical composition comprising a compound of formula (I) substantially as described herein before with a pharmaceutically acceptable diluent or carrier.

Another aspect of the present invention is a compound of formula (I) or a pharmaceutical composition comprising a compound of formula (I) for use in medicine.

Another aspect of the present invention is a compound of formula (I) or a pharmaceutical composition comprising a compound of formula (I) for use in the preparation of a medicament for the treatment of gastrin related disorders.

Typical gastrin related disorders are gastrointestinal ulcers, dyspepsia, reflux oesophagitis (gastroesophageal reflux disease (GERD), both erosive and non-erosive), Zollinger-Ellison syndrome, Barrett's oesophagus (specialized intestinal metaplasia of distal oesophagus), ECL cell hyperplasia, rebound hypersecretion (following cessation of anti-secretory therapy), ECL-derived gastric polyps, cancers of the GI tract, more particularly in the stomach, oesophagus and colo-rectal areas, as well as tumours found in other organs such as the pancreas, lung (small cell lung carcinomas) and thyroid (thyroid medullary tumours) and anxiety. The potentiation of opiate induced analgesia may also provide a role for the gastrin ligands of the present invention.

Yet another aspect of the present invention is a method of making a pharmaceutical composition comprising a compound of formula (I) substantially as described herein before, comprising mixing said compound with a pharmaceutically acceptable diluent or carrier.

Pharmaceutically acceptable salts of the acidic or basic compounds of the invention can of course be made by conventional procedures, such as by reacting the free base or acid with at least a stoichiometric amount of the desired salt-forming acid or base.

Pharmaceutically acceptable salts of the acidic compounds of the invention include salts with inorganic cations such as sodium, potassium, calcium, magnesium, zinc, and ammonium, and salts with organic bases. Suitable organic bases include N-methyl-D-glucamine, arginine, benzathine, diolamine, olamine, procaine, chlorine and tromethamine.

Pharmaceutically acceptable salts of the basic compounds of the invention include salts derived from organic or inorganic acids. Suitable anions include acetate, adipate, besylate, bromide, camsylate, chloride, citrate, edisylate, estolate, fumarate, gluceptate, gluconate, glucuronate, hippurate, hyclate, hydrobromide, hydrochloride. iodide, isethionate, lactate, lactobionate, maleate, mesylate, methylbromide, methylsulfate, napsylate, nitrate, oleate, pamoate, phosphate, polygalacturonate, stearate, succinate, sulfate, sulfosalicylate, tannate, tartrate, terephthalate, tosylate and triethiodide.

It is anticipated that the compounds of the invention can be administered by oral or parenteral routes, including intravenous, intramuscular, intraperitoneal, subcutaneous, rectal and topical administration, and inhalation.

For oral administration, the compounds of the invention will generally be provided in the form of tablets or capsules or as an aqueous solution or suspension.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate and lactose. Corn starch and alginic acid are suitable disintegrating agents. Binding agents may include starch and gelatine. The lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatine capsules in which the active ingredient is mixed with a solid diluent and soft gelatine capsules wherein the active ingredient is mixed with water or an oil such as peanut oil, liquid paraffin or olive oil.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

Effective doses of the compounds of the present invention may be ascertained be conventional methods. The specific dosage level required for any particular patient will depend on a number of factors, including severity of the condition being treated, the route of administration and the weight of the patient. In general, however, it is anticipated that the daily dose (whether administered as a single dose or as divided doses) will be in the range 0.001 to 5000 mg per day, more usually from 1 to 1000 mg per day, and most usually from 10 to 200 mg per day. Expressed as dosage per unit body weight, a typical dose will be expected to be between 0.01 μg/kg and 50 mg/kg, especially between 10 μg/kg and 10 mg/kg, eg. between 100 μg/kg and 2 mg/kg.

In a further aspect of the present invention there are provided pharmaceutical compositions comprising a compound according to formula (I) and a proton pump inhibitor. Compositions comprising a CCK₂/gastrin antagonist and a proton pump inhibitor are described in International patent application WO93/12817, incorporated herein by reference.

In one aspect of the present invention the proton pump inhibitor is
omeprazole which is 5-methoxy-2-[[(4-methoxy-3,5-dimethyl-2-pyridinyl)-methyl]sulfinyl]-1H-benzimidazole;
BY308;
SK&F 95601 which is 2-[[(3-chloro-4-morpholino-2-pyridyl)methyl]sulfinyl]-5-methoxy-(1H)-benzimidazole;
SK & 96067 which is 3-butyryl-4-(2-methylphenylamino)-8-methoxyquinoline;
5-trifluoromethyl-2-[4-methoxy-3-methyl-2-pyridyl-methyl]-thio-[1H]-benzimidazole;
   or pharmaceutically acceptable salts thereof.

These proton pump inhibitors are described and claimed in U.S. Pat. Nos. 4,472,409 and 4,255,431. These patents are incorporated herein by reference.

In a further aspect of the present invention, the proton pump inhibitor is
lansoprazole which is 2-[[[3-methyl-4-(2,2,2-trifluoroethoxy)-2-pridinyl]methyl]sulfinyl]-1H-benzimidazole;
pantoprazole which is 5-(difluoromethoxy)-2-[[(3,4-dimethoxy-2-pyridinyl)methyl]sulfinyl]-1H-benzimidazole;
perprazole;
rabeprazole which is 2-[[4-(3-methoxypropoxy)-3-methylpyridin-2-yl]methylsulfinyl]-1H-benzimidazole;
[[4-(2,2,2-trifluoroethoxy)-3-methyl-2-pyridyl]-methyl]sulfenamide;
(Z)-5-methyl-2-[2-(1-naphthyl)ethenyl]-4-piperidinopyridine HCl;
2-(4-cyclohexyloxy-5-methylpyridin-2-yl)-3-(1-naphthyl)-1-propanol;
methyl 2-cyano-3-(ethylthio)-3-(methylthio)-2-propenoate;
2-((4-methoxy-2-pyridyl)methylsulphinyl)-5-(1,1,2,2-tetrafluoroethoxy)-1H-benzimidazole sodium;
2-[[[4-(2,2,3,3,4,4,4-heptafluorobutoxy)-2-pyridyl]methyl)sulfinyl]-1H-thieno [3,4-d]imidazole;
2-[[[4-(2,2,2-trifluoroethoxy)-3-methyl-2-pyridyl]methyl]sulfinyl]-1H-benzimidazole;
2-[[[4-(2,2,2-trifluoroethoxy)-3-methyl-2-pyridyl]methyl]sulfinyl]-1H-benzimidazole;
2-methyl-8-(phenylmethoxy)-imidazo(1,2-A)-pyridine-3-acetonitrile;
(2-((2-dimethylaminobenzyl)sulfinyl)-benzimidazole);
4-(N-allyl-N-methylamino)-1-ethyl-8-((5-fluoro-6-methoxy-2-benzimidazolyl) sulfinylmethyl)-1-ethyl 1,2,3,4-tetrahydroquinolone;
2-[[(2-dimethylaminophenyl)methyl]sulfinyl]-4,7-dimethoxy-1H-benzimidazole;
2-[(2-(2-pyridyl)phenyl)sulfinyl)-1H-benzimidazole;
(2-[(2-amino-4-methylbenzyl)sulfinyl]-5-methoxybenzo[d]imidazole;
(4(2-methylpyrrol-3-yl)-2-guanidisothiazole);
4-(4-(3-(imidazole)propoxy)phenyl)-2phenylthiazole;
(E)-2-(2-(4-(3-(dipropylamino)butoxy)phenyl)-ethenyl)benzoxazole;
(E)-2-(2-(4-(3-(dipropylamino)propoxy)phenyl)ethenyl)-benzothiazole;
Benzeneamine, 2-[[(5-methoxy-1H-benzimidazol-2-yl)sulfinyl]methyl)-4-methyl-;
Pumilacidin A;
2,3-dihydro-2-methoxycarbonylamino-1,2-benzisothiazol-3-one;
2-(2-ethylanninophenylmethylsulfinyl)-5,6-dimethoxybenzimidazole;
2-methyl-8-(phenylmethoxy)imidazo[1,2-a)pyridine-3-acetonitrile;
3-amino-2-methyl-8-phenylmethoxyimidazo[1,2-a)-pyrazine HCl;
2-[[(3-chloro-4-morpholino-2-pyridyl)methyl]-sulfinyl)-5-methoxy-(1H)-benzinidazole;
[3-butyryl-4-(2-methylphenylamino)-8-methoxy-quinoline);
2-indanyl 2-(2-pyridyl)-2-thiocarbamoylacetate HCl;
2,3-dihydro-2-(2-pyridinyl)-thiazolo (3,2-a)-benzimidazole;
3-cyanomethyl-2-methyl-8-(3-methyl-2-butenyloxy)-(1,2-a)imidazopyridine;
zinc L-carnosine;
   or pharmaceutically acceptable salts thereof.

Rabeprazole is described in U.S. Pat. No. 5,045,552. Lansoprazole is described in U.S. Pat. No. 4,628,098. Pantoprazole is described in U.S. Pat. No. 4,758,579. These patents are incorporated herein by reference.

Preferably, the proton pump inhibitor is selected from (RS)-rabeprazole, (RS)-omeprazole, lansoprazole, pantoprazole, (R)-omeprazole, (S)-omeprazole, perprazole, (R)-rabeprazole, (S)-rabeprazole, or the alkaline salts thereof. The alkaline salts may be, for example, the lithium, sodium, potassium, calcium or magnesium salts.

Compositions of this invention comprising a compound of formula (I) and a proton pump inhibitor may be administered as described above. Preferably the dose of each of the active ingredients in these compositions will be equal to or less than that which is approved or indicated in monotherapy with said active ingredient.

In another aspect of this invention, there is provided a kit comprising a compound of formula (I) and a proton pump inhibitor. The kit is useful as a combined preparation for simultaneous, separate or sequential use in the treatment of patients suffering from gastrointestinal disorders.

In yet a further aspect of the present invention there is provided a method of making a pharmaceutical composition comprising a compound of formula (I) substantially as described herein before and a proton pump inhibitor, comprising mixing said compound and said proton pump inhibitor with a pharmaceutically acceptable carrier or diluent.

The term “hydrocarbyl” is used herein to refer to monovalent groups consisting of carbon and hydrogen. Hydrocarbyl groups thus include alkyl, alkenyl and alkynyl groups (in both straight and branched chain forms), cycloalkyl (including polycycloalkyl groups such as bicyclooctyl and adamantyl), cycloalkenyl and aryl groups, and combinations of the foregoing, such as alkylcycloalkyl, alkylpolycycloalkyl, alkylaryl, alkenylaryl, alkynylaryl, cycloalkylaryl and cycloalkenylaryl groups.

Where reference is made to a carbon atom of a hydrocarbyl group being replaced by a N, O or S atom, what is intended is that 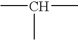
is replaced by 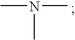
or that —CH₂— is replaced by —O— or —S—.

Where reference is made to an optionally substituted hydrocarbyl group, the hydrocarbyl group is substituted with 1, 2 or 3 groups independently selected from -L-Q

wherein:

L is a bond, or a group of the formula —(CR¹⁷R¹⁸)ᵥ—Y—(CR¹⁷R¹⁸)_{w}, wherein v and w are independently 0, 1, 2 or 3, and Y is a bond, —CR¹⁵═CR¹⁶—, phenyl, furanyl, thiophenyl, pyrrolyl, thiazolyl, imidazolyl, oxazolyl, isoxazolyl, pyrazolyl, isoxazolonyl, piperazinyl, piperidinyl, morpholinyl, pyrrolidinyl, isothiazolyl, triazolyl, oxadiazolyl, thiadiazolyl, pyridyl or pyridazyl;

Q is H, (C₁ to C₆ alkyl)oxy, [N-Z](C₁ to C₆ alkyl)oxy(C₁ to C₆ alkyl)amino, thio, (C₁ to C₆ alkyl)thio, carboxy(C₁ to C₆ alkyl)thio, carboxy, carboxy(C₁ to C₆ alkyl), carboxy(C₁ to C₆ alkenyl), [N-Z]carboxy(C₁ to C₆ alkyl)amino, carboxy(C₁ to C₆ alkyl)oxy, formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, amino, [N-Z](C₁ to C₆ alkyl)amino, aminocarbonyl, (C₁ to C₆ alkyl)aminocarbonyl, di(C₁ to C₆ alkyl)aminocarbonyl, [N-Z](C₁ to C₆ alkyl)carbonylamino, C₅ to C₈ cycloalkyl, [N-Z](C₁ to C₆ alkyl)carbonyl(C₁ to C₆ alkyl)amino, halo, halo(C₁ to C₆ alkyl), sulfamoyl, [N-Z](C₁ to C₆ alkyl)sulfonylamino, (C₁ to C₆ alkyl)sulfonylaminocarbonyl, carboxy(C₁ to C₆ alkyl)sulfonyl, carboxy(C₁ to C₆ alkyl)sulfinyl, tetrazolyl, [N-Z]tetrazolylamino, cyano, amidino, amidinothio, SO₃H, formyloxy, formamido, C₃ to C₈ cycloalkyl, (C₁ to C₆ alkyl)sulphamoyl, di(C₁ to C₆ alkyl)sulphamoyl, (C₁ to C₆ alkyl)carbonylaminosulfonyl, 5-oxo-2,5-dihydro[1,2,4]oxadiazolyl, carboxy(C₁ to C₆ alkyl)carbonylamino, tetrazolyl(C₁ to C₆ alkyl)thio, [N-Z]tetrazolyl(C₁ to C₆ alkyl)amino, 5-oxo-2,5-dihydro[1,2,4]thiadiazolyl, 5-oxo-1,2-dihydro[1,2,4]triazolyl, [N-Z](C₁ to C₆ alkyl)amino(C₁ to C₆ alkyl)amino, or a group of the formula 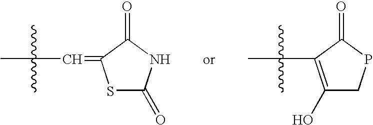
wherein P is O, S or NR¹⁹;
and

Z is H, C₁ to C₆ alkyl, t-butoxycarbonyl, acetyl, benzoyl or benzyl.

The term “alkyl” is used herein to refer to both straight and branched chain forms. Further, the alkyl chain may include multiple bonds. Hence, the term “alkyl” also encompasses alkenyl and alkynyl groups. Likewise, the term “cycloalkyl” also encompasses cycloalkenyl groups. Preferably, alkyl and cycloalkyl groups as used in the present invention do not contain multiple bonds. Where there are preferred alkenyl groups, these are specified as alkenyl groups. However, specific reference to alkenyl groups is not to be construed as any limitation on the definition of alkyl groups as described above.

Where reference is made to dialkyl groups [e.g. di(C₁ to C₆ alkyl)amino groups], it is understood that the two alkyl groups may be the same or different.

In the interests of simplicity, terms which are normally used to refer to monovalent groups (such as “alkyl” or “phenyl”) are also used herein to refer to divalent bridging groups which are formed from the corresponding monovalent group by the loss of one hydrogen atom. Whether such a term refers to a monovalent group or to a divalent group will be clear from the context. For example, when L is —(CR¹⁷R¹⁸)ᵥ—Y—(CR¹⁷R¹⁸)_{w}—, it is clear that Y must be a divalent group. Thus, when Y is defined as thiazolyl, for example, this refers to a divalent group having the structure

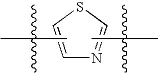

Where, as in this example, a divalent bridging group is formed from a cyclic moiety, the linking bonds may be on any suitable ring atom, subject to the normal rules of valency. Accordingly, by way of further example, the term pyrrolyl in the definition of Y includes all of the following groups:

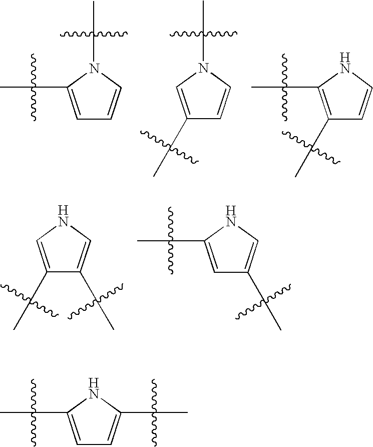

The term “halogen” or “halo” is used herein to refer to any of fluorine, chlorine, bromine and iodine. Most usually, however, halogen substituents in the compounds of the invention are chlorine and fluorine substituents. Groups such as halo(C₁ to C₆ alkyl) includes mono-, di- or tri-halo substituted C₁ to C₆ alkyl groups. Moreover, the halo substitution may be at any position in the alkyl chain.

The prefix [N-Z] refers to possible substitution of an amino group in the following compound or substituent name. For example, [N-Z]alkylamino refers to groups of the form

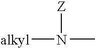

Similarly, [N-Z]tetrazolylamino, wherein Z is C₁ to C₆ alkyl, includes groups such as tetrazolyl[N-methyl]amino and tetrazolyl[N-ethyl]amino. Of course, when Z is H, no substitution is present.

In case there is any doubt, the group named as 5-oxo-2,5-dihydro[1,2,4]oxadiazolyl has the following formula 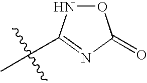
and comprehends tautomeric forms.

The invention is now further illustrated by means of the following Examples.

Experimental

All reactions were performed under an atmosphere of dry argon unless otherwise stated. Commercially available dichloromethane (DCM), tetrahydrofuran (THF) and N,N-dimethylformamide (DMF) were used. In reactions in which anilines were used, where necessary un-reacted aniline was removed either by chromatography or by stirring with excess methylisocyanate polystyrene HL resin (200-400 mesh, 2 mmol/g) in DCM (R. J. Booth, et. al., J. Am. Chem. Soc., (1997), 119, 4882). Flash column chromatography was performed on Merck silica gel 60 (40-63 μm) using the reported solvent systems. ¹H NMR spectra were recorded on a Bruker DRX-300 instrument at 300 MHz and the chemical shifts (δ_{H}) were recorded relative to an internal standard. (2-Amino-phenyl)-cyclohexyl-methanone was prepared by a published method (M. S. Chambers, et. al., Bioorg Med. Chem. Lett. (1993), 3, 1919), and (2-amino-phenyl)-cyclopentyl-methanone and 1-(2-amino-phenyl)-3-methyl-butan-1-one were prepared by a modification of this method. 2-Bromo-1-cyclopentyl-ethanone and 2-bromo-1-cyclohexyl-ethanone were prepared by a published method (M. Gaudry, A. Marquet, Org. Synth., (1976), 55, 24), and 2-bromo-1-cyclopropyl-ethanone was prepared by a modification of this method. 2-Bromo-1-(1-methyl-cyclopentyl)-ethanone was prepared by a published method (T. S. Sorensen, J. Am. Chem. Soc., (1969), 91, 6398). Substituted anilines were either obtained commercially, synthesized by the literature method indicated where first mentioned or prepared in a number of steps and from the starting material as indicated, using standard chemical transformations.

### EXAMPLE 1

### 2-[2,4-Dioxo-1-(2-oxo-2-pyrrolidin-1-yl-ethyl)-5-phenyl-1,2,4,5-tetrahydro-1,3,5-benzotriazepin-3-yl]-N-phenyl-acetamide

Step a. 2,4-Dioxo-1-(2-oxo-2-pyrrolidin-1-yl-ethyl)-5-phenyl-1,2,4,5-tetrahydro-1,3,5-benzotriazepine. 2-(2-Phenylamino-phenylamino)-1-pyrrolidin-1-yl-ethanone (700 mg, 2.37 mmol) (Bioorg. Med. Chem. Lett. 1997, 7, 281) was dissolved in anhydrous dimethylacetamide (DMA) (40 ml) and the mixture was heated to 90° C. A DMA solution (3 ml) of phenyl isocyanatoformate (600 mg, 3.68 mmol) (Tetrahedron, 1975, 31, 2007) was added dropwise to the latter and the mixture was stirred overnight at 90° C. After cooling, the reaction mixture was partitioned between ethyl acetate (25 ml) and brine (50 ml). The organic layer was separated and washed successively with further brine (2×50 ml) then dried (MgSO₄). Concentration of the solvents afforded an oil, which yielded a white solid after column chromatography (gradient 5:1 dichloromethane/ethyl acetate to neat ethyl acetate) (430 mg, 49%). ¹H (DMSO-d₆) 9.08 (1H, s), 7.52-7.23 (6H, m), 7.18 (1H, m), 7.11 (1H, m), 7.74 (1H, d), 4.72 (2H, m), 3.51 (2H, m), 3.32 (2H, m), 1.92 (2H, m), 1.77 (2H, m).

Step b. [2,4-Dioxo-1-(2-oxo-2-pyrrolidin-1-yl-ethyl)-5-phenyl-1,2,4,5-tetrahydro-1,3,5-benzotriazepin-3-yl]-acetic acid tert-butyl ester. The product of Example 1 Step a (140 mg, 0.38 mmol) and sodium hydride (22 mg, 60% suspension in mineral oil, 0.55 mmol) were stirred in anhydrous DMF (10 ml) for 0.5 h. Thereafter, tert-butyl bromoacetate (112 mg, 0.57 mmol) was added and the reaction mixture was allowed to stir at ambient temperature overnight, then cautiously poured into a water/ethyl acetate mixture (1:1, 25 ml). The organic layer was separated, washed with water (2×20 ml) and brine (2×20 ml) and dried (MgSO₄). Concentration afforded the crude product, which was purified by chromatography (ethyl acetate/dichloromethane 1:1) to afford a beige solid (165 mg, 90%).

¹H (CDCl₃) 7.54 (2H, d), 7.42 (3H, m), 7.37 (1H, t), 7.17 (1H, dt), 7.07 (1H, dt), 6.84 (1H, d), 4.67 (1H, d), 4.59 (1H, d), 4.25 (1H, d), 4.13 (1H, d), 3.54 (4H, m), 2.01 (2H, m), 1.87 (2H, m), 1.40 (9H, s).

Step c. [2,4-Dioxo-1-(2-oxo-2-pyrrolidin-1-yl-ethyl)-5-phenyl-1,2,4,5-tetrahydro-1,3,5-benzotriazepin-3-yl]-acetic acid. The product of the previous example (165 mg, 0.34 mmol) was stirred in neat TFA (4 ml) at ambient temperature for 1 h. The solution was then concentrated in vacuo to afford an oil, which was redissolved in dichloromethane (10 ml) and reconcentrated. The resulting oil was redissolved in dichloromethane, and the organic layer was washed with water and dried (MgSO₄). Concentration afforded an orange paste (140 mg, 96%). ¹H (CDCl₃) 10.29 (1H, brs), 7.47-7.34 (6H, m), 7.27 (1H, t), 7.10 (1H, t), 6.86 (1H, m), 4.67 (2H, m), 4.25 (2H, m), 3.59 (4H, m), 2.06 (2H, m), 1.94 (2H, m).

Step d. 2-[2,4-Dioxo-1-(2-oxo-2-pyrrolidin-1-yl-ethyl)-5-phenyl-1,2,4,5-tetrahydro-1,3,5-benzotriazepin-3-yl]-N-phenyl-acetamide. The product of Example 1 Step c (150 mg, 0.35 mmol), EDC (95 mg, 0.50 mmol), HOBT (68 mg, 0.50 mmol), triethylamine (120 μl, 0.87 mmol), DMAP (10 mg, catalytic amount) and aniline (40 mg, 0.43 mmol) were combined in DMF (5 ml) and stirred overnight at ambient temperature. The reaction mixture was poured into ethyl acetate (20 ml) and washed successively with water (2×20 ml) and brine (2×20 ml) then dried (MgSO₄). Concentration afforded the crude product, which yielded an off-white solid from dichloromethane/hexane (75 mg, 43%). ¹H (CDCl₃) 8.49 (1H, brs), 7.54 (2H, d), 7.43-7.33 (9H, m), 7.18 (1H, m), 7.12 (1H, m), 6.88 (1H, d), 4.79 (1H, d), 4.60 (1H, d), 4.32 (2H, s), 3.53 (4H, m), 1.99-1.84 (4H, m). Found: C 65.23, H 5.83, N 13.74%; C₂₈H₂₇N₅O₄.H₂O requires: C 65.23, H 5.67, N 13.58%.

### EXAMPLE 2

### 2-[2,4-Dioxo-1-(2-oxo-2-pyrrolidin-1-yl-ethyl)-5-phenyl-1,2,4,5-tetrahydro-1,3,5-benzotriazepin-3-yl]-N-(4-fluoro-phenyl)-acetamide

The title product was prepared using essentially the same procedure as in Example 1 Step d, except that [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1, Step c) was reacted with 4-fluoroaniline instead of aniline. The title product was obtained as a white solid. ¹H (CDCl₃) 8.54 (1H, brs), 7.56-7.36 (7H, m), 7.31-7.27 (3H, m), 6.95- 6.88 (3H, d), 4.75 (1H, d), 4.60 (1H, d), 4.29 (2H, m), 3.55 (4H, m), 2.06 (2H, m), 1.89 (2H, m). Found: C 62.81, H 5.52, N 13.08%; C₂₈H₂₆FN₅O₄.H₂O requires: C 63.03, H 5.29, N 13.12%.

### EXAMPLE 3

### 3-{2-[2,4-Dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid

Step a. 3-{2-[2,4-Dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid benzyl ester.

The title product was prepared using essentially the same procedure as in Example 1 Step d, except that [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1, Step c) was reacted with 3-amino-benzoic acid benzyl ester instead of aniline. ¹H (CDCl₃) 8.67 (1H, brs), 7.85-7.73 (3H, m), 7.53-7.21 (14H, m), 6.90 (1H, m), 5.34 (2H, s), 4.68 (2H, m), 4.30 (2H, m), 3.54 (4H, m), 2.04 (2H, m), 1.91 (2H, m).

Step b. The product of Step a (126 mg, 0.20 mmol) was stirred with 10% Pd/C (20 mg) in a THF/MeOH (1:1, 10 ml) mixture under a hydrogen atmosphere overnight. The reaction mixture was filtered over a pad of Celite and washed through the filter pad with CH₂Cl₂ (20 ml), and then concentrated in vacuo. The title product was obtained as a white solid from dichloromethane/hexane. ¹H (DMSO-d₆) 10.24 (1H, brs), 8.19 (1H, s), 7.69-7.17 (12H, m), 6.83 (1H, d), 4.91 (1H, d), 4.70 (1H, d), 4.26 (2H, m), 3.44 (4H, m), 1.90 (2H, m), 1.77 (2H, m). Found m/z, 541; C₂₉H₂₇N₅O₆ requires: 541.

### EXAMPLE 4

[5-Cycloheptyl-2,4-dioxo-3-(m-tolylcarbamoyl-methyl)-1,2,4,5-tetrahydro-1,3,5-benzotriazepin-1-yl]-acetic acid benzyl ester

Step a. (2-Cycloheptylamino-phenylamino)-acetic acid benzyl ester. A DMF (20 ml) mixture of N-cycloheptyl-benzene-1,2-diamine (Bioorg. Med. Chem. Lett. 1997, 7, 281) (1 g, 5.0 mmol), K₂CO₃ (690 mg, 50 mmol) and 2-benzyl bromoacetate (1.15 g, 5.0 mmol) was stirred overnight at ambient temperature. After dilution with water (20 ml) and ethyl acetate (20 ml), the organic layer was separated and washed successively with water (2×20 ml) and brine (2×20 ml) then dried (MgSO₄). Concentration afforded a red oil (1.73 g, 98%). ¹H (CDCl₃) 7.38 (5H, m), 6.82 (1H, dd), 6.75 (1H, dt), 6.62 (1H, dd), 6.58 (1H, dd), 5.22 (2H, s), 3.94 (2H, s), 3.42 (1H, m), 3.40 (1H, m), 1.97 (2H, m), 1.71-1.56 (10H, m).

Step b. (5-Cycloheptyl-2,4-dioxo-1,2,4,5-tetrahydro-1,3,5-benzotriazepin-1-yl)-acetic acid benzyl ester. This was made using essentially the same procedure as in Example 1 Step a except that the product of the previous step was used instead of 2-(2-phenylamino-phenylamino)-1-pyrrolidin-1-yl-ethanone. ¹H (CDCl₃) 7.61-7.23 (9H, m), 6.31 (1H, s), 5.20 (2H, s), 4.60 (1H, d), 4.47 (1H, d), 3.93 (1H, m), 2.18-1.21 (12H, m).

Step c. (1-Benzyloxycarbonylmethyl-5-cycloheptyl-2,4-dioxo-1,2,4,5-tetrahydro-1,3,5-benzotriazepin-3-yl)-acetic acid. This was made using essentially the same procedures as in Example 1 Steps b and c except that 5-cycloheptyl-2,4-dioxo-1,2,4,5-tetrahydro-1,3,5-benzotriazepin-1-yl)-acetic acid benzyl ester was used in place of 2,4-dioxo-1-(2-oxo-2-pyrrolidin-1-yl-ethyl)-5-phenyl-1,2,4,5-tetrahydro-1,3,5-benzotriazepine. ¹H (CDCl₃) 10.15 (1H, brs), 7.37-7.24 (9H, m), 5.19 (2H, m), 4.62 (1H, d), 4.44 (1H, d), 4.17 (2H, s), 3.93 (1H, m), 2.19-1.44 (12H, m).

Step d. The title product was prepared using essentially the same procedure as in Example 1 Step d, except that the product of the previous step was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1 Step c) and that m-toluidine was used instead of aniline. ¹H (CDCl₃) 8.21 (1H, brs), 7.61-7.29 (7H, m), 7.22 (2H, m), 6.87 (1H, m), 5.19 (2H, m), 4.64 (1H, d), 4.46 (1H, d), 4.20 (2H, s), 3.98 (1H, m), 2.26 (3H, s), 2.25-1.70 (12H, m). Found: C 66.70, H 6.27, N 9.21%; C₃₃H₃₆N₄O₅.1.3 H₂O requires: C 66.94, H 6.57, N 9.46%.

### EXAMPLE 5

[5-Cycloheptyl-2,4-dioxo-3-(m-tolylcarbamoyl-methyl)-1,2,4,5-tetrahydro-1,3,5-benzotriazepin-1-yl]-acetic acid

The title compound was prepared using essentially the same procedure as in Example 3 Step b except that [5-cycloheptyl-2,4-dioxo-3-(m-tolylcarbamoyl-methyl)-1,2,4,5-tetrahydro-1,3,5-benzotriazepin-1-yl]-acetic acid benzyl ester (Example 4, Step d) was used instead of 3-{2-[2,4-dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid benzyl ester. ¹H (CDCl₃) 8.31 (1H, brs), 7.38-7.29 (3H, m), 7.23-7.14 (4H, m), 6.87 (1H, m), 4.52 (2H, m), 4.20 (2H, m), 4.02 (1H, m), 2.25 (3H, m), 2.21-1.40 (12H, m). The product was converted into its N-methyl-D-glucamine) salt and lyophilised from dioxan/water. Found: C 54.41, H 6.99, N 8.85%; C₂₆H₃₀N₄O₅.0.9 CH₂Cl₂/0.7 dioxan requires: C 54.29, H 6.75, N 8.63%.

### EXAMPLE 6

2-[1-Cycloheptyl-2,4-dioxo-5-(2-oxo-2-pyrrolidin-1-yl-ethyl)-1,2,4,5-tetrahydro-1,3,5-benzotriazepin-3-yl]-N-m-tolyl-acetamide

The title product was prepared using essentially the same procedure as in Example 1 Step d, except that [5-cycloheptyl-2,4-dioxo-3-(m-tolylcarbamoyl-methyl)-1,2,4,5-tetrahydro-1,3,5-benzotriazepin-1-yl]-acetic acid (Example 5) was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1 Step c) and pyrrolidine was used instead of aniline. ¹H (CDCl₃) 8.24 (1H, brs), 7.41-7.14 (7H, m), 6.86 (1H, m), 4.48 (2H, s), 4.21 (2H, m), 4.04 (1H, m), 3.49 (4H, m), 2.35 (3H, s), 2.30-1.51 (16H, m). Found: C 66.21, H 7.09, N 10.98%; C₃₀H₃₇N₅O₄.1EtOAc requires: C 65.89, H 7.31, N 11.30%.

### EXAMPLE 7

### 2-[1-Cycloheptyl-5-(3,3-dimethyl-2-oxo-butyl)-2,4-dioxo-1,2,4,5-tetrahydro-1,3,5-benzotriazepin-3-yl]-N-m-tolyl-acetamide

Step a. 1-(2-Cycloheptylamino-phenylamino)-3,3-dimethyl-butan-2-one. This was made using essentially the same procedure as in Example 4 Step a except that 1-bromo-3,3-dimethyl-butan-2-one was used instead of 2-benzyl bromoacetate.

¹H (CDCl₃) 6.77 (2H, m), 6.61 (2H, m), 4.32 (1H, brs), 4.11 (2H, s), 3.43 (1H, m), 3.27 (1H, m), 1.72 (1H, m), 1.62-1.41 (12H, m), 1.27 (9H, s).

Step b. 1-Cycloheptyl-5-(3,3-dimethyl-2-oxo-butyl)-2,4-dioxo-1,2,4,5-tetrahydro-1,3,5-benzotriazepine. This was made using essentially the same procedure as in Example 1 Step a except that the product of the previous step was used instead of 2-(2-phenylamino-phenylamino)-1-pyrrolidin-1 -yl-ethanone.

¹H (CDCl₃) 7.32-7.19 (3H, m), 7.00 (1H, m), 6.23 (1H, brs), 4.78 (1H, d), 4.67 (1H, d), 3.96 (1H, m), 2.25 (2H, m), 1.94-1.79 (8H, m), 1.29 (9H, s).

Step c. [1-Cycloheptyl-5-(3,3-dimethyl-2-oxo-butyl)-2,4-dioxo-1,2,4,5-tetrahydro-1,3,5-benzotriazepin-3-yl]-acetic acid benzyl ester. This was made using essentially the same procedure as in Example 1 Step b except that the product of the previous step was used instead of 2,4-dioxo-1-(2-oxo-2-pyrrolidin-1-yl-ethyl)-5-phenyl-1,2,4,5-tetrahydro-1,3,5-benzotriazepine and benzyl 2-bromoacetate was used instead of tert-butyl bromoacetate.

Step d. [1-Cycloheptyl-5-(3,3-dimethyl-2-oxo-butyl)-2,4-dioxo-1,2,4,5-tetrahydro-1,3,5-benzotriazepin-3-yl]-acetic acid. This was prepared using essentially the same procedure as in Example 3 Step b except that the product of the previous step was used instead of 3-{2-[2,4-dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid benzyl ester. ¹H (CDCl₃) 7.30-7.17 (3H, m), 7.04 (1H, m), 4.74 (2H, m), 3.99 (3H, m), 2.28-1.52 (12H, m), 1.27 (9H, s).

Step e. The title product was made using essentially the same procedure as in Example 1 Step d except that the product of the previous step was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1 Step c) and m-toluidine was used instead of aniline. ¹H (CDCl₃) 8.20 (1H, brs), 7.39-7.10 (7H, m), 6.86 (1H, m), 4.77 (2H, m), 4.20 (2H, m), 4.05 (1H, m), 2.36 (3H, s), 2.30-1.74 (12H, m), 1.25 (9H, s). Found: C 68.29, H 7.22, N 10.23%; C₃₀H₃₈N₄O₄.0.1CH₂Cl₂ requires: C 68.58, H 7.30, N 10.63%.

### EXAMPLE 8

### (3-{2-[1-Cycloheptyl-5-(3,3-dimethyl-2-oxo-butyl)-2,4-dioxo-1,2,4,5-tetrahydro-1,3,5-benzotriazepin-3-yl]-acetylamino}-phenyl)-acetic acid

Step a. (3-{2-[1-Cycloheptyl-5-(3,3-dimethyl-2-oxo-butyl)-2,4-dioxo-1,2,4,5-tetrahydro-1,3,5-benzotriazepin-3-yl]-acetylamino}-phenyl)-acetic acid benzyl ester. This was prepared using essentially the same procedure as in Example 1 Step d except that [1-cycloheptyl-5-(3,3-dimethyl-2-oxo-butyl)-2,4-dioxo-1,2,4,5-tetrahydro-1,3,5-benzotriazepin-3-yl]-acetic acid (Example 7 Step d) was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1 Step c) and (3-amino-phenyl)-acetic acid benzyl ester was used instead of aniline. ¹H (CDCl₃) 8.20 (1H, brs), 7.37-7.27 (11H, m), 7.20 (1H, m), 7.07 (1H, m), 5.12 (2H, s), 4.77 (1H, d), 4.70 (1H, d), 4.18 (1H, d), 4.13 (1H, d), 4.09 (1H, m), 3.61 (2H, s), 2.04-1.32 (11H, m), 1.26 (9H, s).

Step b. The title product was made using essentially the same procedure as in Example 3 Step b except that the product of the previous step was used instead of 3-{2-[2,4-dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid benzyl ester. ¹H (CDCl₃) 8.20 (1H, brs), 7.34-7.21 (5H, m), 7.20 (1H, m), 7.19 (1H, m), 6.97 (1H, m), 4.74 (1H, d), 4.63 (1H, d), 4.17 (2H, m), 4.00 (1H, m), 3.46 (2H, s), 1.89-1.54 (12H, m), 1.24 (9H, s). The product was converted into its (N-methyl-D-glucamine) salt and lyophilised from dioxan/water. Found: C 57.91, H 7.59, N 7.97%; C₃₈H₄₄N₄O₆.1.4H₂O/1.1 dioxan requires: C 57.87, H 7.62, N 7.96%.

### EXAMPLE 9

### (3-{2-[5-Cycloheptyl-5-(3,3-dimethyl-2-oxo-butyl)-2,4-dioxo-1,2,4,5-tetrahydro-1,3,5-benzotriazepin-3-yl]-acetylamino}-phenylsulfanyl)-acetic acid ethyl ester

The title product was prepared using essentially the same procedure as in Example 1 Step d except that [1-cycloheptyl-5-(3,3-dimethyl-2-oxo-butyl)-2,4-dioxo-1,2,4,5-tetrahydro-1,3,5-benzotriazepin-3-yl]-acetic acid (Example 7 Step d) was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1 Step c) and (3-amino-phenylsulfanyl)-acetic acid ethyl ester (Bioorg. Med Chem., 1997, 5, 1433) was used instead of aniline. ¹H (CDCl₃) 8.34 (1H, brs), 7.44-7.16 (7H, m), 7.08 (1H, m), 4.75 (2H, m), 4.18 (4H, m), 4.14 (1H, m), 3.64 (2H, s), 2.20-1.56 (12H, m), 1.25 (12H, m).

### EXAMPLE 10

### (3-{2-[]-Cycloheptyl-5-(3,3-dimethyl-2-oxo-butyl)-2,4-dioxo-1,2,4,5-tetrahydro-1,3,5-benzotriazepin-3-yl]-acetylamino}-phenylsulfanyl)-acetic acid benzyl ester

Step a. (3-amino-phenylsulfanyl)-acetic acid benzyl ester. This was made using essentially the same procedure as for (3-amino-phenylsulfanyl)-acetic acid ethyl ester (Bioorg. Med. Chem., 1997, 5 1433) except that benzyl 2-bromoacetate was used instead of ethyl bromoacetate. ¹H (CDCl₃) 7.34-7.18 (8H,m), 7.05 (1H,m), 6.34 (1H,brs), 5.16 (2H,s), 3.68 (2H,s), 1.53 (9H,s).

Step b. The title product was prepared using essentially the same procedure as in Example 1 Step d except that [1-cycloheptyl-5-(3,3-dimethyl-2-oxo-butyl)-2,4-dioxo-1,2,4,5-tetrahydro-1,3,5-benzotriazepin-3-yl]-acetic acid (Example 7 Step d) was used instead of [2,4-dioxo-1-(2-oxo-2-pyrrolidin-1-yl-ethyl)-5-phenyl- 1,2,4,5-tetrahydro-1,3,5-benzotriazepin-3-yl]-acetic acid (Example 1 Step c) and (3-amino-phenylsulfanyl)-acetic acid benzyl ester was used instead of aniline. ¹H (CDCl₃) 8.33 (1H, brs), 7.40-7.30 (10H, m), 7.27-7.05 (3H, m), 5.14 (2H, s), 4.74 (2H, m), 4.16 (2H, m), 4.05 (1H, m), 4.00 (2H, s), 2.20-1.58 (12H, m), 1.29 (12H, m).

### EXAMPLE 11

### (3-{2-[1-Cycloheptyl-5-(3,3-dimethyl-2-oxo-butyl)-2,4-dioxo-1,2,4,5-tetrahydro-1,3,5-benzotriazepin-3-yl]-acetylamino}-phenylsulfanyl)-acetic acid

(3-{2-[1-cycloheptyl-5-(3,3-dimethyl-2-oxo-butyl)-2,4-dioxo-1,2,4,5-tetrahydro-1,3,5-benzotriazepin-3-yl]-acetylamino}-phenylsulfanyl)-acetic acid benzyl ester (Example 10) (120 mg, 0.17 mmol) and NaOH (0.25 ml of a 1N solution, 0.25 mmol) were stirred overnight in MeOH (5 ml) at ambient temperature. KHSO₄ (2 ml, 10% solution) was added and the resulting white precipitate was collected by filtration, then redissolved in CH₂Cl₂ and dried (MgSO₄). Concentration and addition of hexane afforded a white solid, which was collected by filtration and dried in vacuo (80 mg, 77%). ¹H (CDCl₃) 8.50 (1H, brs), 7.39-7.28 (6H, m), 7.15-7.05 (3H, m), 4.80 (1H, m), 4.70 (1H, m), 4.17 (2H, s), 4.03 (1H, m), 3.62 (2H, s), 2.29-1.56 (12H, m), 1.26 (12H, m). The product was converted into its (N-methyl-D-glucamine) salt and lyophilised from dioxan/water. Found: C 53.52, H 6.97, N 7.89%; C₃₈H₅₅N₅O₁₁S.3.4H₂O requires: C 53.62, H 7.32, N 8.23%.

### EXAMPLE 12

### 2-[1-Cycloheptyl-5-(3,3-dimethyl-2-oxo-butyl)-2,4-dioxo-1,2,4,5-tetrahydro-1,3,5-benzotriazepin-3-yl]-N-[3-(5-oxo-2,5-dihydro-[1,2,4]-oxadiazol-3-yl)-phenyl]-acetamide

The title compound was synthesised using essentially the same procedure as in Example 1, Step d except that [1-cycloheptyl-5-(3,3-dimethyl-2-oxo-butyl)-2,4-dioxo-1,2,4,5-tetrahydro-1,3,5-benzotriazepin-3-yl]-acetic acid (Example 7 Step d) was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1 Step c) and 3-(3-amino-phenyl)-2H-[1,2,4]oxadiazol-5-one (PCT GB93/00535) was used instead of aniline. ¹H (CDCl₃) 10.80 (1H, brs), 9.15 (1H, s), 8.02 (1H, s), 7.57-7.07 (8H, m), 4.85 (1H, d), 4.64 (1H, d), 4.31 (2H, s), 4.10 (1H, m), 1.72-1.25 (12H, m), 1.20 (9H, s). The product was converted into its (N-methyl-D-glucamine) salt and lyophilised from dioxan/water. Found: C 55.98, H 6.77, N 10.91%; C₃₈H₅₃N₇O₁₁.1.4H₂O/0.7 dioxan requires: C 56.28, H 7.10, N 11.26%.

### EXAMPLE 13

### 2-[1-Cyclohexyl-5-(3,3-dimethyl-2-oxo-butyl)-2,4-dioxo-1,2,4,5-tetrahydro-1,3,5-benzotriazepin-3-yl]-N-(3-methylamino-phenyl)-acetamide

Step a. 1-(2-Cyclohexylamino-phenylamino)-3,3-dimethyl-butan-2-one. This was made using essentially the same procedure as in Example 4 Step a except that N-cyclohexyl-benzene-1,2-diamine was used instead of N-cycloheptyl-benzene-1,2-diamine and 1-bromo-3,3-dimethyl-butan-2-one was used instead of 2-benzyl bromoacetate. ¹H (CDCl₃) 6.78-6.71 (3H, m), 6.57 (1H, m), 4.20 (1H, brs), 4.10 (2H, s), 3.43 (1H, m), 3.30 (2H, m), 2.09 (2H, m), 1.82-1.76 (3H, m), 1.41-1.25 (14H, m).

Step b. 1-Cyclohexyl-5-(3,3-dimethyl-2-oxo-butyl)-2,4-dioxo-1,2,4,5-tetrahydro-1,3,5-benzotriazepine. This was made using essentially the same procedure as in Example 1 Step a except that 1-(2-cyclohexylamino-phenylamino)-3,3-dimethyl-butan-2-one was used instead of 2-(2-phenylamino-phenylamino)-1-pyrrolidin-1-yl-ethanone. ¹H (CDCl₃) 7.37 (1H,m), 7.27 (2H,m), 6.99 (1H,m), 6.20 (1H,brs), 4.73 (2H,m), 3.80 (1H,m), 2.20-1.35 (7H,m), 1.34-1.26 (12H,m).

Step c. [1-Cyclohexyl-5-(3,3-dimethyl-2-oxo-butyl)-2,4-dioxo-1,2,4,5-tetrahydro-1,3,5-benzotriazepin-3-yl]-acetic acid benzyl ester. This was made using essentially the same procedure as in Example 1 Step b except that the product of the previous step was used instead of 2,4-dioxo-1-(2-oxo-2-pyrrolidin-1-yl-ethyl)-5-phenyl-1,2,4,5-tetrahydro-1,3,5-benzotriazepine and benzyl 2-bromoacetate was used instead of tert-butyl bromoacetate.

¹H (CDCl₃) 7.33-7.18 (8H,m), 6.99 (1H,m), 5.09 (2H,m), 4.76 (1H,d), 4.68 (1H,d), 4.33 (1H,d), 4.23 (1H,d), 3.85 (1H,m), 2.18 (1H,m), 1.91-1.34 (6H,m), 1.33-1.25 (11H,m).

Step d. [1-Cyclohexyl-5-(3,3-dimethyl-2-oxo-butyl)-2,4-dioxo-1,2,4,5-tetrahydro-1,3,5-benzotriazepin-3-yl]-acetic acid. This was made using essentially the same procedure as in Example 3 Step b except that the product of the previous step was used instead of 3-{2-[2,4-dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid benzyl ester. ¹H (CDCl₃) 10.40 (1H,brs), 7.36 (1H,m), 7.25 (2H, m), 7.03 (1H, m), 4.75 (2H, m), 4.05 (2H,s), 3.85 (1H, m), 2.27 (1H,m), 1.99-1.65 (6H, m), 1.43-1.20 (12H, s).

Step e. (3-{2-[1-Cyclohexyl-5-(3,3-dimethyl-2-oxo-butyl)-2,4-dioxo-1,2,4,5-tetrahydro-1,3,5-benzotriazepin-3-yl]-acetylamino}-phenyl)-methyl-carbamic acid tert-butyl ester.

This was made using essentially the same procedure as in Example 1, Step d except that the product of the previous step was used instead of 2-[2,4-dioxo-1-(2-oxo-2-pyrrolidin-1-yl-ethyl)-5-phenyl-1,2,4,5-tetrahydro-1,3,5-benzotriazepin-3-yl]-N-phenyl-acetamide (Example 1, Step c) and (3-amino-phenyl)-methyl-carbamic acid tert-butyl ester was used instead of aniline. ¹H (CDCl₃) 8.30 (1H, brs), 7.43-7.27 (4H,m), 7.19-7.10 (3H,m), 6.95 (1H,d), 4.78 (1H,d), 4.73 (1H,d), 4.16 (2H,m), 3.90 (1H,m), 3.23 (3H,s), 2.28 (1H,m), 1.89-1.45 (6H,m), 1.45 (9H,s), 1.34-1.25 (12H,m).

Step f. The title product was made using essentially the same procedure as in Example 1 Step c except that the product from the previous step was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid tert-butyl ester (Example 1 step b). ¹H (CDCl₃) 8.10 (1H, brs), 7.42 (1H,m), 7.28 (2H,m), 7.11-7.01 (3H,m), 6.45 (1H,m), 6.36 (1H,d), 4.78 (1H,d), 4.67 (1H,d), 4.19 (2H,m), 3.90 (1H,m), 2.81 (3H,s), 2.28 (1H,m), 1.83-1.38 (6H,m), 1.35-1.21 (12H,m).

### EXAMPLE 14

### 3-(3-{2-[1-Cyclohexyl-5-(3,3-dimethyl-2-oxo-butyl)-2,4-dioxo-1,2,4,5-tetrahydro-1,3,5-benzotriazepin-3-yl]-acetylamino}-phenyl)-propionic acid

Step a. 3-(3-{2-[1-Cyclohexyl-5-(3,3-dimethyl-2-oxo-butyl)-2,4-dioxo-1,2,4,5-tetrahydro-1,3,5-benzotriazepin-3-yl]-acetylamino}-phenyl)-propionic acid tert-butyl ester. This was made using essentially the same procedure as in Example 1 Step d except that [1-cyclohexyl-5-(3,3-dimethyl-2-oxo-butyl)-2,4-dioxo-1,2,4,5-tetrahydro-1,3,5-benzotriazepin-3-yl]-acetic acid (Example 13 Step d) was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1 Step c) and 3-(3-amino-phenyl)-propionic acid tert-butyl ester was used instead of aniline.

Step b. The title product was made using essentially the same procedure as in Example 1 Step c except that the product from the previous step was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid tert-butyl ester (Example 1 Step b). ¹H (CDCl₃) 8.79 (1H,brs), 7.41 (1H,m), 7.40-7.28 (5H,m), 7.10 (1H,m), 6.94 (1H,m), 4.85 (1H,d), 4.68 (1H, d), 4.15 (2H,s), 3.89 (1H,m), 2.90 (2H,m), 2.62 (2H,m), 2.20 (1H,m), 1.79-1.39 (6H,m), 1.34-1.27 (12H,m). The product was converted into its (N-methyl-D-glucamine) salt and lyophilised from dioxan/water. Found: C 54.75, H 6.82, N 7.82%; C₃₈H₅₅N₅O₁₁.1.2 CH₂Cl₂/0.4 H₂O requires: C 54.84, H 6.53, N 7.91%.

### EXAMPLE 15

### 2-[1-Cyclohexyl-5-(3,3-dimethyl-2-oxo-butyl)-2,4-dioxo-1,2,4,5-tetrahydro-1,3,5-benzotriazepin-3-yl]-N-[3-(2-methyl-thiazol-4-yl)-phenyl]-acetamide

This was made by using essentially the same procedure as in Example 1, Step d except that [1-cyclohexyl-5-(3,3-dimethyl-2-oxo-butyl)-2,4-dioxo-1,2,4,5-tetrahydro-1,3,5-benzotriazepin-3-yl]-acetic acid (Example 13 Step d) was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1 Step c) and 3-(2-methyl-thiazol-4-yl)-phenylamine was used instead of aniline. ¹H (CDCl₃) 8.27 (1H,brs), 7.80 (1H,s), 7.46 (1H,d), 7.30 (3H,m), 7.28 (3H,m), 7.08 (1H,m), 4.83 (1H,d), 4.70 (1H,d), 4.21 (2H,m), 3.91 (1H,m), 2.89 (3H,s), 2.31 (1H,m), 1.94-1.85 (5H,m), 1.41 (1H,m), 1.35 (12H,m). Found: C 64.24, H 6.05, N 11.85%; C₃₂H₃₇N₅O₄.0.2 CH₂Cl₂ requires: C 63.95, H 6.23, N 11.58%.

### EXAMPLE 16

### 2-[1-Cyclohexyl-5-(3,3-dimethyl-2-oxo-butyl)-2,4-dioxo-1,2,4,5-tetrahydro-1,3,5-benzotriazepin-3-yl]-N-{3-[methyl-(2H-tetrazol-5-yl)-amino]-phenyl}-acetamide

This was made by using essentially the same procedure as in Example 1, Step d except that [1-cyclohexyl-5-(3,3-dimethyl-2-oxo-butyl)-2,4-dioxo-1,2,4,5-tetrahydro-1,3,5-benzotriazepin-3-yl]-acetic acid (Example 13 Step d) was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1 Step c) and N-methyl-N-(2H-tetrazol-5-yl)-benzene-1,3-diamine (J. Med. Chem. 1996, 39, 842) was used instead of aniline. ¹H (CDCl₃) 10.50 (1H,brs), 8.50 (1H,brs), 7.36 (2H,m), 7.26 (4H,m), 7.02 (1H,m), 7.00 (1H,m), 4.79 (1H,d), 4.67 (1H,d), 3.89 (1H,m), 3.55 (3H,s), 2.20 (1H,m), 1.96-1.63 (6H,m), 1.36-1.23 (12H,m).

### EXAMPLE 17

### 2-[1-Cyclohexyl-5-(3,3-dimethyl-2-oxo-butyl)-2,4-dioxo-1,2,4,5-tetrahydro-1,3,5-benzotriazepin-3-yl]-N-[3-(5-oxo-2,5-dihydro-[1,2,4]oxadiazol-3-yl)-phenyl]-acetamide

The title compound was made using essentially the same procedure as in Example 1, Step d except that [1-cyclohexyl-5-(3,3-dimethyl-2-oxo-butyl)-2,4-dioxo-1,2,4,5-tetrahydro-1,3,5-benzotriazepin-3-yl]-acetic acid (Example 13 Step d) was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1, Step c) and 3-(3-amino-phenyl)-2H-[1,2,4]oxadiazol-5-one (PCT GB93/00535) was used instead of aniline. ¹H (CDCl₃) 9.15 (1H, s), 7.57-7.07 (9H, m), 4.82 (1H, d), 4.63 (1H, d), 4.31 (2H, m), 3.89 (1H, m), 2.17 (1H,m), 1.84-1.64 (6H, m), 1.27-1.15 (12H, m). The product was converted into its (N-methyl-D-glucamine) salt and lyophilised from dioxan/water. Found: C 53.39, H 6.38, N 11.59%; C₃₇H₅₁N₇O₁₁.1 CH₂Cl₂ requires: C 53.39, H 6.25, N 11.47%.

### EXAMPLE 18

### (6-{2-[1-Cyclohexyl-5-(3,3-dimethyl-2-oxo-butyl)-2,4-dioxo-1,2,4,5-tetrahydro-1,3,5-benzotriazepin-3-yl]-acetylamino}-indol-1-yl)-acetic acid ethyl ester

The title compound was made using essentially the same procedure as in Example 1, Step d except that [1-cyclohexyl-5-(3,3-dimethyl-2-oxo-butyl)-2,4-dioxo-1,2,4,5-tetrahydro-1,3,5-benzotriazepin-3-yl]-acetic acid (Example 13 Step d) was used instead of [2,4-dioxo-1-(2-oxo-2-pyrrolidin-1-yl-ethyl)-5-phenyl-1,2,4,5-tetrahydro-1,3,5-triazepin-3-yl]-acetic acid (Example 1 Step c) and (6-amino-indol-1-yl)-acetic acid ethyl ester (made from 6-nitro-1H-indole in two steps) was used instead of aniline. ¹H (CDCl₃) 8.13 (1H, brs), 7.90 (1H,s), 7.44 (2H,m), 7.32 (2H,m), 7.11 (1H,m), 7.09 (1H,m), 6.57 (1H,m), 6.48 (1H,m), 4.79 (4H,m), 4.24 (2H,m), 3.90 (1H, m), 3.74 (2H,m), 2.29 (1H,m), 1.87-1.34 (7H, m), 1.30-1.20 (14H, m). Found: C 65.18, H 6.71, N 11.19%; C₃₄H₄₁N₅O₆.0.2 CH₂Cl₂ requires: C 64.92, H 6.60, N 11.07%.

### EXAMPLE 19

### 4-[1-Cyclohexyl-5-(3,3-dimethyl-2-oxo-butyl)-2,4-dioxo-1,2,4,5-tetrahydro-1,3,5-benzotriazepin-3-yl]-N-[3-(5-oxo-2,5-dihydro-[1,2,4]oxadiazol-3-yl)-phenyl]-butyramide

Step a. [1-Cyclohexyl-5-(3,3-dimethyl-2-oxo-butyl)-2,4-dioxo-1,2,4,5-tetrahydro-1,3,5-benzotriazepin-3-yl]-butyric acid benyl ester. A DMF (10 ml) solution of 1-cyclohexyl-5-(3,3-dimethyl-2-oxo-butyl)-2,4-dioxo-1,2,4,5-tetrahydro-1,3,5-benzotriazepine (Example 13 Step b) (250 mg, 0.70 mmol) was stirred with NaH (40 mg, 60% suspension in mineral oil, 1 mmol) for 0.5 h at ambient temperature. Benzyl-4-bromobutyrate (J. Med. Chem. 1996, 39, 5176) (300 mg, 1.17 mmol) was added and the mixture was stirred at 60° C. for 72 h. After cooling, the reaction mixture was poured into ethyl acetate/water (1:1, 25 ml) and the organic layer was separated and washed with brine (2×20 ml) then water (2×20 ml) and dried (MgSO₄). Solvent concentration afforded the crude product, which was purified by chromatography (gradient 20:1-5:1CH₂Cl₂/EtOAc) to afford an oil (210 mg, 57%). ¹H (CDCl₃) 7.34-7.29 (5H, m), 7.27-7.16 (3H,m), 6.96 (1H,m), 5.01 (2H,s), 4.68 (2H, m), 3.83 (1H, m), 3.51 (2H,m), 2.15-1.75 (8H,m), 1.66-1.18 (11H, m).

Step b. [1-cyclohexyl-5-(3,3-dimethyl-2-oxo-butyl)-2,4-dioxo-1,2,4,5-tetrahydro-1,3,5-benzotriazepin-3-yl]-butyric acid. This was made using essentially the same procedure as in Example 3, Step b except that the product of the previous step was used instead of 3-{2-[2,4-dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid benzyl ester (Example 3, Step a). ¹H (CDCl₃) 7.32-7.18 (3H, m), 6.97 (1H,m), 4.68 (2H, m), 3.76-3.71 (3H, m), 3.50 (2H,m), 2.07-1.80 (8H,m), 1.70-1.23 (11H, m).

Step c. The title compound was made using essentially the same procedure as in Example 1, Step d except that the product of the previous step was used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid and 3-(3-amino-phenyl)-2H-[1,2,4]oxadiazol-5-one (PCT GB93/00535) was used instead of aniline. ¹H (CDCl₃) 8.65 (1H, s), 7.89 (1H,s), 7.68 (1H,m), 7.53 (1H,m), 7.37-7.20 (5H, m), 6.94 (1H,m), 4.90 (1H, d), 4.52 (1H, d), 3.84 (1H, m), 3.51 (2H,m), 2.18-1.47 (9H,m), 1.35-1.26 (16H, m). The product was converted into its (N-methyl-D-glucamine) salt and lyophilised from dioxan/water. Found: C 56.52, H 6.75, N 11.40%; C₃₉H₅₅N₇O₁₁.1.5 H₂O.0.1 dioxan requires: C 56.76, H 7.10, N 11.76%.

### EXAMPLE 20

### 3-{2-[]-Cyclohexyl-5-(2-cyclopentyl-2-oxo-ethyl)-2,4-dioxo-1,2,4,5-tetrahydro-1,3,5-benzotriazepin-3-yl]-acetylamino}-benzoic acid

Step a. 2-(2-Cyclohexylamino-phenylamino)-1-cyclopentyl-ethanone. This is made using essentially the same procedure as in Example 4 Step a except that N-cyclohexyl-benzene-1,2-diamine is used instead of N-cycloheptyl-benzene-1,2-diamine and 2-bromo-1-cyclopentyl-ethanone is used instead of 2-benzylbromoacetate.

Step b. 1-Cyclohexyl-5-(2-cyclopentyl-2-oxo-ethyl)-2,4-dioxo-1,2,4,5-tetrahydro-1,3,5-benzotriazepine. This is made using essentially the same procedure as in Example 1 Step a except that the product of the previous step is used instead of 2-(2-phenylamino-phenylamino)-1-pyrrolidin-1-yl-ethanone.

Step c. [1-Cyclohexyl-5-(2-cyclopentyl-2-oxo-ethyl)-2,4-dioxo-1,2,4,5-tetrahydro-1,3,5-benzotriazepin-3-yl]-acetic acid benzyl ester. This is made using essentially the same procedure as in Example 1 Step b except that the product of the previous step is used instead of 2,4-dioxo-1-(2-oxo-2-pyrrolidin-1-yl-ethyl)-5-phenyl-1,2,4,5-tetrahydro-1,3,5-benzotriazepine and benzyl 2-bromoacetate is used instead of tert-butyl bromoacetate.

Step d. [1-Cyclohexyl-5-(2-cyclopentyl-2-oxo-ethyl)-2,4-dioxo-1,2,4,5-tetrahydro-1,3,5-benzotriazepin-3-yl]-acetic acid. This is made using essentially the same procedure as in Example 3 Step b except that the product of the previous step is used instead of 3-{2-[2,4-dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid benzyl ester.

Step e. (3-{2-[1-Cyclohexyl-5-(2-cyclopentyl-2-oxo-ethyl)-2,4-dioxo-1,2,4,5-tetrahydro-1,3,5-benzotriazepin-3-yl]-acetylamino}-phenyl)-methyl-carbamic acid tert-butyl ester. This is made using essentially the same procedure as in Example 1 Step d except that the product of the previous step is used instead of 2-[2,4-dioxo-1-(2-oxo-2-pyrrolidin-1-yl-ethyl)-5-phenyl-1,2,4,5-tetrahydro-1,3,5-benzotriazepin-3-yl]-N-phenyl-acetamide (Example 1 Step c) and (3-amino-phenyl)-methyl-carbamic acid tert-butyl ester is used instead of aniline.

Step f. The title product is made using essentially the same procedure as in Example 1 Step c except that the product from the previous step is used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid tert-butyl ester (Example 1 Step b).

### EXAMPLE 21

### 3-{2-[1-Cyclohexyl-5-(2-cyclohexyl-2-oxo-ethyl)-2,4-dioxo-1,2,4,5-tetrahydro-1,3,5-benzotriazepin-3-yl]-acetylamino}-benzoic acid

Step a. 2-(2-Cyclohexylamino-phenylamino)-1-cyclohexyl-ethanone. This is made using essentially the same procedure as in Example 4, Step a except that N-cyclohexyl-benzene-1,2-diamine is used instead of N-cycloheptyl-benzene-1,2-diamine and 2-bromo-1-cyclohexyl-ethanone is used instead of 2-benzylbromoacetate.

Step b. 1-Cyclohexyl-5-(2-cyclohexyl-2-oxo-ethyl)-2,4-dioxo-1,2,4,5-tetrahydro-1,3,5-benzotriazepine. This is made using essentially the same procedure as in Example 1 Step a except that the product of the previous step is used instead of 2-(2-phenylamino-phenylamino)-1-pyrrolidin-1-yl-ethanone.

Step c. [1-Cyclohexyl-5-(2-cyclohexyl-2-oxo-ethyl)-2,4-dioxo-1,2,4,5-terahydro-1,3,5-benzotriazepin-3-yl]-acetic acid benzyl ester. This is made using essentially the same procedure as in Example 1, Step b except that the product of the previous step is used instead of 2,4-dioxo-1-(2-oxo-2-pyrrolidin-1-yl-ethyl)-5-phenyl-1,2,4,5-tetrahydro-1,3,5-benzotriazepine and benzyl 2-bromoacetate is used instead of tert-butyl bromoacetate.

Step d. [1-Cyclohexyl-5-(2-cyclohexyl-2-oxo-ethyl)-2,4-dioxo-1,2,4,5-tetrahydro-1,3,5-benzotriazepin-3-yl]-acetic acid. This is made using essentially the same procedure as in Example 3 Step b except that the product of the previous step is used instead of 3-{2-[2,4-dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid benzyl ester.

Step e. (3-{2-[1-Cyclohexyl-5-(2-cyclohexyl-2-oxo-ethyl)-2,4-dioxo-1,2,4,5-tetrahydro-1,3,5-benzotriazepin-3-yl]-acetylamino}-phenyl)-methyl-carbamic acid tert-butyl ester. This is made using essentially the same procedure as in Example 1, Step d except that the product of the previous step is used instead of 2-[2,4-dioxo-1-(2-oxo-2-pyrrolidin-1-yl-ethyl)-5-phenyl-1,2,4,5-tetrahydro-1,3,5-benzotriazepin-3-yl]-N-phenyl-acetamide (Example 1, Step c) and (3-amino-phenyl)-methyl-carbamic acid tert-butyl ester is used instead of aniline.

Step f. The title product is made using essentially the same procedure as in Example 1 Step c except that the product from the previous step is used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid tert-butyl ester (Example 1 Step b).

### EXAMPLE 22

### 3-{2-[1-Cyclohexyl-5-(1-methyl-cyclopentyl-2-oxo-ethyl)-2,4-dioxo-1,2,4,5-tetrahydro-1,3,5-benzotriazepin-3-yl]-acetylamino}-benzoic acid

Step a. 2-(2-Cyclohexylamino-phenylamino)-1-(1-methyl-cyclopentyl)-ethanone.

This is made using essentially the same procedure as in Example 4 Step a except that N-cyclohexyl-benzene-1,2-diamine is used instead of N-cycloheptyl-benzene-1,2-diamine and 2-bromo-1-(1-methyl-cyclopentyl)-ethanone is used instead of 2-benzylbromoacetate.

Step b. 1-Cyclohexyl-5-(1-methyl-cyclopentyl-2-oxo-ethyl)-2,4-dioxo-1,2,4,5-tetrahydro-1,3,5-benzotriazepine. This is made using essentially the same procedure as in Example 1 Step a except that the product of the previous step is used instead of 2-(2-phenylamino-phenylamino)-1-pyrrolidin-1-yl-ethanone.

Step c. [1-Cyclohexyl-5-((1-methyl-cyclopentyl)-2-oxo-ethyl)-2,4-dioxo-1,2,4,5-tetrahydro-1,3,5-benzotriazepin-3-yl]-acetic acid benzyl ester. This is made using essentially the same procedure as in Example 1 Step b except that the product of the previous step is used instead of 2,4-dioxo-1-(2-oxo-2-pyrrolidin-1-yl-ethyl)-5-phenyl-1,2,4,5-tetrahydro-1,3,5-benzotriazepine and benzyl 2-bromoacetate is used instead of tert-butyl bromoacetate.

Step d. [1-Cyclohexyl-5-((1-methyl-cyclopentyl)-2-oxo-ethyl)-2,4-dioxo-1,2,4,5-tetrahydro-1,3,5-benzotriazepin-3-yl]-acetic acid. This is made using essentially the same procedure as in Example 3 Step b except that the product of the previous step is used instead of 3-{2-[2,4-dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid benzyl ester.

Step e. (3-{2-[1-Cyclohexyl-5-((1-methyl-cyclopentyl)-2-oxo-ethyl)-2,4-dioxo-1,2,4,5-tetrahydro-1,3,5-benzotriazepin-3-yl]-acetylamino}-phenyl)-methyl-carbamic acid tert-butyl ester. This is made using essentially the same procedure as in Example 1 Step d except that the product of the previous step is used instead of 2-[2,4-dioxo-1-(2-oxo-2-pyrrolidin-1-yl-ethyl)-5-phenyl-1,2,4,5-tetrahydro-1,3,5-benzotriazepin-3-yl]-N-phenyl-acetamide (Example 1, Step c) and (3-amino-phenyl)-methyl-carbamic acid tert-butyl ester is used instead of aniline.

Step f. The title product is made using essentially the same procedure as in Example 1 Step c except that the product from the previous step is used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid tert-butyl ester (Example 1 Step b).

### EXAMPLE 23

### 3-{2-[1-Cyclohexyl-5-(1-methyl-cyclohexyl-2-oxo-ethyl)-2,4-dioxo-1,2,4,5-tetrahydro-1,3,5-benzotriazepin-3-yl]-acetylamino}-benzoic acid.

Step a. 2-(2-Cyclohexylamino-phenylamino)-1-(1-methyl-cyclohexyl)-ethanone. This is made using essentially the same procedure as in Example 4 Step a except that N-cyclohexyl-benzene-1,2-diamine is used instead of N-cycloheptyl-benzene-1,2-diamine and 2-bromo-1-(1-methyl-cyclohexyl)-ethanone is used instead of 2-benzylbromoacetate.

Step b. 1-Cyclohexyl-5-(1-methyl-cyclohexyl-2-oxo-ethyl)-2,4-dioxo-1,2,4,5-tetrahydro-1,3,5-benzotriazepine. This is made using essentially the same procedure as in Example 1, Step a except that the product of the previous step is used instead of 2-(2-phenylamino-phenylamino)-1-pyrrolidin-1-yl-ethanone.

Step c. [1-Cyclohexyl-5-((1-methyl-cyclohexyl)-2-oxo-ethyl)-2,4-dioxo-1,2,4,5-tetrahydro-1,3,5-benzotriazepin-3-yl]-acetic acid benzyl ester. This is made using essentially the same procedure as in Example 1 Step b except that the product of the previous step is used instead of 2,4-dioxo-1-(2-oxo-2-pyrrolidin-1-yl-ethyl)-5-phenyl-1,2,4,5-tetrahydro-1,3,5-benzotriazepine and benzyl 2-bromoacetate is used instead of tert-butyl bromoacetate.

Step d. [1-Cyclohexyl-5-((1-methyl-cyclohexyl)-2-oxo-ethyl)-2,4-dioxo-1,2,4,5-tetrahydro-1,3,5-benzotriazepin-3-yl]-acetic acid. This is made using essentially the same procedure as in Example 3 Step b except that the product of the previous step is used instead of 3-{2-[2,4-dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid benzyl ester.

Step e. (3-{2-[1-Cyclohexyl-5-((1-methyl-cyclohexyl)-2-oxo-ethyl)-2,4-dioxo-1,2,4,5-tetrahydro-1,3,5-benzotriazepin-3-yl]-acetylamino}-phenyl)-methyl-carbamic acid tert-butyl ester. This is made using essentially the same procedure as in Example 1 Step d except that the product of the previous step is used instead of 2-[2,4-dioxo-1-(2-oxo-2-pyrrolidin-1-yl-ethyl)-5-phenyl-1,2,4,5-tetrahydro-1,3,5-benzotriazepin-3-yl]-N-phenyl-acetamide (Example 1, Step c) and (3-amino-phenyl)-methyl-carbamic acid tert-butyl ester is used instead of aniline.

Step f. The title product is made using essentially the same procedure as in Example 1 Step c except that the product from the previous step is used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid tert-butyl ester (Example 1 Step b).

### EXAMPLE 24

### (3-{2-[1-Cycloheptyl-5-(2-cyclopentyl-2-oxo-ethyl)-2,4-dioxo-1,2,4,5-tetrahydro-1,3,5-benzotriazepin-3-yl]-acetylamino}-phenyl)-acetic acid

Step a. 2-(2-Cycloheptylamino-phenylamino)-1-cyclopentyl-ethanone. This is made using essentially the same procedure as in Example 4 Step a except that 2-bromo-1-cyclopentyl-ethanone is used instead of 2-benzylbromoacetate.

Step b. 1-Cycloheptyl-5-(2-cyclopentyl-2-oxo-ethyl)-2,4-dioxo-1,2,4,5-tetrahydro-1,3,5-benzotriazepine. This is made using essentially the same procedure as in Example 1 Step a except that the product of the previous step is used instead of 2-(2-phenylamino-phenylamino)-1-pyrrolidin-1-yl-ethanone.

Step c. [1-Cycloheptyl-5-(2-cyclopentyl-2-oxo-ethyl)-2,4-dioxo-1,2,4,5-tetrahydro-1,3,5-benzotriazepin-3-yl]-acetic acid benzyl ester. This is made using essentially the same procedure as in Example 1 Step b except that the product of the previous step is used instead of 2,4-dioxo-1-(2-oxo-2-pyrrolidin-1-yl-ethyl)-5-phenyl-1,2,4,5-tetrahydro-1,3,5-benzotriazepine and benzyl 2-bromoacetate is used instead of tert-butyl bromoacetate.

Step d. [1-Cycloheptyl-5-(2-cyclopentyl-2-oxo-ethyl)-2,4-dioxo-1,2,4,5-tetrahydro-1,3,5-benzotriazepin-3-yl]-acetic acid. This is made using essentially the same procedure as in Example 3 Step b except that the product of the previous step is used instead of 3-{2-[2,4-dioxo-1-(2-oxo-2-pyrrolidin-1-yl-ethyl)-5-phenyl-1,2,4,5-tetrahydro-1,3,5-benzotriazepin-3-yl]-acetylamino}-benzoic acid benzyl ester.

Step e. (3-{2-[1-Cycloheptyl-5-(2-cyclopentyl-2-oxo-ethyl)-2,4-dioxo-1,2,4,5-tetrahydro-1,3,5-benzotriazepin-3-yl]-acetylamino}-phenyl)-methyl-carbamic acid tert-butyl ester. This is made using essentially the same procedure as in Example 1 Step d except that the product of the previous step is used instead of 2-[2,4-dioxo-1-(2-oxo-2-pyrrolidin-1-yl-ethyl)-5-phenyl-1,2,4,5-tetrahydro-1,3,5-benzotriazepin-3-yl]-N-phenyl-acetamide (Example 1 Step c) and (3-amino-phenyl)-methyl-carbamic acid tert-butyl ester is used instead of aniline.

Step f. The title product is made using essentially the same procedure as in Example 1 Step c except that the product from the previous step is used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid tert-butyl ester (Example 1 Step b).

### EXAMPLE 25

### 3-(3-{2-[1-Cycloheptyl-5-(2-cyclopentyl-2-oxo-ethyl)-2,4-dioxo-1,2,4,5-tetrahydro-1,3,5-benzotriazepin-3-yl]-acetylamino}-phenyl)-propionic acid

Step a. 3-(3-{2-[1-Cycloheptyl-5-(2-cyclopentyl-2-oxo-ethyl)-2,4-dioxo-1,2,4,5-tetrahydro-1,3,5-benzotriazepin-3-yl]-acetylamino}-phenyl)-propionic acid tert-butyl ester. This is made using essentially the same procedure as in Example 1 Step d except that [1-cycloheptyl-5-(2-cyclopentyl-2-oxo-ethyl)-2,4-dioxo-1,2,4,5-tetrahydro-1,3,5-benzotriazepin-3-yl]-acetic acid (Example 24 Step d) is used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1 Step c) and 3-(3-amino-phenyl)-propionic acid tert-butyl ester is used instead of aniline.

Step b. The title product is made using essentially the same procedure as in Example 1 Step c except that the product from the previous step is used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid tert-butyl ester Example 1 step b).

### EXAMPLE 26

### 2-[1-Cycloheptyl-5-(2-cyclopentyl-2-oxo-ethyl)-2,4-dioxo-1,2,4,5-tetrahydro-1,3,5-benzotriazepin-3-yl]-N-[3-(1H-tetrazol-5-yl)-phenyl]-acetamide

The title compound is made using essentially the same procedure as in Example 1 Step d except that [1-cycloheptyl-5-(2-cyclopentyl-2-oxo-ethyl)-2,4-dioxo-1,2,4,5-tetrahydro-1,3,5-benzotriazepin-3-yl]-acetic acid (Example 24 step d) is used instead of [2,4-dioxo-1-(2-oxo-2-pyrrolidin-1-yl-ethyl)-5-phenyl-1,2,4,5-tetrahydro-1,3,5-benzotriazepin-3-yl]-acetic acid (Example 1 Step c) and 2,2-dimethyl-propionic acid 5-(3-amino-phenyl)-tetrazol-1-ylmethyl ester (J. Med. Chem. 1996, 39, 842) is used instead of aniline. This is followed by removal of the protecting group using methanolic ammonia (p625, T. W. Greene, P. G. Wuts, Protective Groups in Synthesis, 1999, 3^{rd} ed. John Wiley & Sons).

### EXAMPLE 27

### 2-[1-Cycloheptyl-5-(2-cyclopentyl-2-oxo-ethyl)-2,4-dioxo-1,2,4,5-tetrahydro-1,3,5-triazepin-3-yl]-N-[3-(5-oxo-2,5-dihydro-[1,2,4]oxadiazol-3-yl)\α-phenyl]-acetamide

The title compound is made using essentially the same procedure as in Example 1 Step d except that [1-cycloheptyl-5-(2-cyclopentyl-2-oxo-ethyl)-2,4-dioxo-1,2,4,5-tetrahydro-1,3,5-benzotriazepin-3-yl]-acetic acid (Example 24 step d) is used instead of [2,4-dioxo-1-(2-oxo-2-pyrrolidin-1-yl-ethyl)-5-phenyl-1,2,4,5-tetrahydro-1,3,5-triazepin-3-yl]-acetic acid (Example 1 Step c) and 3-(3-amino-phenyl)-2H-[1,2,4]oxadiazol-5-one is used instead of aniline.

CCK₂ and CCK₁ Antagonist Activity

Certain compounds of the examples were tested for gastrin (CCK₂) antagonist activity in an immature rat stomach assay. The procedure was as follows:

The oesophagus of immature rats (33-50 g, ca. 21 days old) was ligated at the level of the cardiac sphincter and the duodenal sphincter was cannulated. The stomach was excised and flushed with ca. 1 ml of unbuffered physiological saline solution. The fundus was punctured and cannulated. A further 4-5 ml of unbuffered solution was flushed through the stomach to ensure the preparation was not leaking. The stomach was lowered into a jacketed organ bath containing 40 ml of buffered solution containing 3×10⁻⁸ M 5-methylfurmethide, maintained at 37° and gassed vigorously with 95% O₂/5% CO₂. The stomach was continuously perfused at a rate of 1 ml min⁻¹ with unbuffered solution gassed with 100% O₂ with the perfusate passing over an internally referenced pH-electrode fixed 12 cm above the stomach.

After 120 min of stabilisation the drugs were added directly to the serosal solution in the organ bath and after a further 60 min cumulative pentagastrin dose-response curves were started. Changes in acid secretion were monitored and the curves analysed according to Black et al., Br. J. Pharmacol., 1985, 86, 581.

The CCK₁ activity of the ligands was assessed in a radioligand binding study, looking at the displacement of [³H]-L-364,718 from sites in CHO-K1 cells into which the human CCK₁-receptor sequence has been cloned. Data is shown in the Table. S.E.M's on pKᵢ are ±≦0.1

The results obtained at CCK₂ and CCK₁ receptors are set out in the following Table.

In vitro biological activity of 1,3,5-benzotriazepines

| Example | CCK₂ pK_{B} ± sem | CCK₁ pK_{I} |
|---|---|---|
| 2 | 5.65 ± 0.32 | 5.1 |
| 3 | 6.15 ± 0.21 | <5.0 |
| 8 | 7.36 ± 0.21 | 5.4 |
| 10 | 8.17 ± 0.30 | 7.0 |
| 11 | 8.35 ± 0.33 | 6.6 |
| 12 | 8.49 ± 0.33 | 6.9 |
| 13 | 7.71 ± 0.33 | 5.6 |
| 14 | 7.58 ± 0.42 | 5.6 |
| 15 | 7.70 ± 0.35 | 5.8 |
| 16 | 7.90 ± 0.31 | 5.7 |
| 17 | 7.35 ± 0.28 | |
| 19 | 7.06 ± 0.35 | |

It is found that the compositions and products of the present invention comprising a compound of formula (I) and a proton pump inhibitor reduce hyperplasia, associated with administration of proton pump inhibitors. This was measured according to the following experimental protocol.

Animals and Treatment:

40 male SPF Wistar rats (200 g) were divided into 4 treatment groups and 2 strata. The treatment of the 20 rats in the second stratum started 2 weeks after the treatment of the first stratum. The design of the study was completely randomised double blind with individual blinding; all rats were placed in a separate cage. Animals had continuous access to water and food.

Animals were treated once daily during 14 days:
Control group: 1 ml gastrin test drug vehicle+1 ml p.o.(gavage) 0,25% Methocel (Dow Corning)
PPI group: 1 ml gastrin test drug vehicle+1 ml p.o.(gavage) 25 mg/kg Rabeprazole in 0.25% Methocel.
GRA group: 1 ml gastrin test drug+1 ml p.o. (gavage) 0,25% Methocel
GRA-PPI group: 1 ml gastrin test drug+1 ml p.o.(gavage) 25 mg/kg Rabeprazole in 0.25% Methocel.

Gastrin test drug made up to an appropriate dose in physiologically compatible solvent.

Preparation of Tissue:

After removal of the fundus, the stomach were rinsed with phosphate buffered saline prior to fixation with 4% formalin in Millonig buffer. After 4 hours immersion in fixative solutions at room temperature, tissue was rinsed in phosphate buffered saline (PBS), dehydrated and embedded in paraffin using the Leitz paraffin embedding station (Leitz TP 1050; Germany) dehydration module and paraffin embedding module (Leitz EG 1160; Germany).

Cross sections (3 μm thick) of the oxyntic part of the stomach were made at 3 levels, each separated by a distance of 400 μm.

Immunostaining

The following indirect immunofluorescence labeling method was used:
removal of paraffin and rehydratation of the sections followed by a blocking step
primary antibodies: polyclonal guinea pig anti-histidine decarboxylase, 1/2000 (from Euro-Diagnostica) and monoclonal mouse anti PCNA 1/2500 (Clone PC10 from Sigma). All antibodies were diluted in a 0.2% BSA solution. Sections were incubated overnight at 4° C. and then washed with a BSA solution.
secondary antibodies: goat anti guinea pig coupled to CY5, 1/500 (from Jackson Laboratories) and goat anti-mouse coupled to Cy3, 1/250 (from Jackson Laboratories); incubation for 4 hours at 37° C. After rinsing with BSA and PBS solutions, sections were mounted with slowfade (Molecular Probes Europe BV), and stored at 4° C.
   Imaging

Fluorescence labelling was observed with an epifluorescence microscope or a Zeiss LSM510 (Carl Zeiss Jena GmbH) confocal microscope.

By using CY5- and CY3-coupled antibodies, the high autofluorescence properties of the oxyntic mucosa were circumvented when sections are illuminated by a 488 nm (FITC channel) light source. Negative controls, by omitting the primary antibodies, and an isotype control staining for PCNA showed complete absence of staining. The specific labelling of PCNA was checked using double staining with TOPRO-3® (Molecular Probes Europe BV), a nuclear stain. Only in the most luminal located epithelial cells, non-specific cytoplasmic labelling was present. In the glandular part of the mucosa, non-specific PCNA-staining was absent.

For determination of the labelling index of ECL cells, at least 80 confocal images per rat were taken from the 3 slides at the 3 different levels. The ratio of double labelled cells (HDC+PCNA) and all HDC labelled cells yielded the labelling index of ECL cells.

Proliferation activity of ECL cells in the PPI group is expected to be increased compared with sham, GRA and GRA-PPI groups (Eissele, R., Patberg, H., Koop, H., Krack, W., Lorenz, W., McKnight, A. T., and Arnold, R. Effect of gastrin receptor blockade on endrocine cells in rats during achlorhydria. Gastroenterology, 103, 1596-1601, 1992). Increased proliferation by PPI will be completely blocked by GRA.

1. A compound of formula (I) 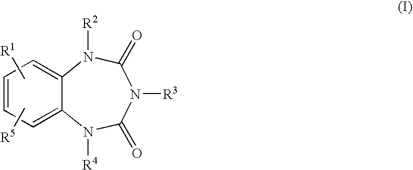
wherein:
R¹ and R⁵ are independently H, C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino, (C₁ to C₆ alkyl)aminocarbonyl, di(C₁ to C₆ alkyl)aminocarbonyl, [N-Z](C₁ to C₆ alkyl)carbonylamino, formyloxy, formamido, (C₁ to C₆ alkyl)aminosulfonyl, di(C₁ to C₆ alkyl)aminosulfonyl, [N-Z](C₁ to C₆ alkyl)sulfonylamino or cyano; or R¹ and R⁵ together form a methylenedioxy group;
R² is an optionally substituted C₁ to C₁₈ hydrocarbyl group wherein up to three C atoms may optionally be replaced by N, O and/or S atoms;
R³ is —(CR¹¹R¹²)ₘ—X—(CR¹³R¹⁴)ₚ-R⁹;
m is 0, 1, 2, 3 or 4;
p is 0, 1 or 2;
X is a bond, —CR¹⁵═CR¹⁶—, —C≡C—, C(O)NH, NHC(O), C(O)NMe, NMeC(O), C(O)O, NHC(O)NH, NHC(O)O, OC(O)NH, NH, O, CO, SO₂, SO₂NH, C(O)NHNH, 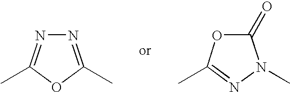
R⁹ is H; C₁ to C₆ alkyl; or phenyl, naphthyl, pyridyl, benzimidazolyl, indazolyl, quinolinyl, isoquinolinyl, tetrahydroisoquinolinyl, indolinyl, isoindolinyl, indolyl, isoindolyl or 2-pyridonyl, all optionally substituted with 1,2 or 3 groups independently selected from
-L-Q
wherein:
L is a bond, or a group of the formula —(CR¹⁷R¹⁸)ᵥ—Y—(CR¹⁷R¹⁸)_{w}, wherein v and w are independently 0, 1, 2 or 3, and Y is a bond, —CR¹⁵═CR¹⁶—, phenyl, furanyl, thiophenyl, pyrrolyl, thiazolyl, imidazolyl, oxazolyl, isoxazolyl, pyrazolyl, isoxazolonyl, piperazinyl, piperidinyl, morpholinyl, pyrrolidinyl, isothiazolyl, triazolyl, oxadiazolyl, thiadiazolyl, pyridyl or pyridazyl; and
Q is H, (C₁ to C₆ alkyl)oxy, [N-Z](C₁ to C₆ alkyl)oxy(C₁ to C₆ alkyl)amino, thio, (C₁ to C₆ alkyl)thio, carboxy(C₁ to C₆ alkyl)thio, carboxy, carboxy(C₁ to C₆ alkyl), carboxy(C₁ to C₆ alkenyl), [N-Z]carboxy(C₁ to C₆ alkyl)amino, carboxy(C₁ to C₆ alkyl)oxy, formyl, (C₁ to C₆ alkyl)carbonyl, (C₁ to C₆ alkyl)oxycarbonyl, (C₁ to C₆ alkyl)oxycarbonyl(C₁ to C₆ alkyl)thio, (C₁ to C₆ alkyl)carbonyloxy, nitro, trihalomethyl, hydroxy, amino, [N-Z](C₁ to C₆ alkyl)amino, aminocarbonyl, (C₁ to C₆ alkyl)aminocarbonyl, di(C₁ to C₆ alkyl)aminocarbonyl, [N-Z](C₁ to C₆ alkyl)carbonylamino, C₅ to C₈ cycloalkyl, [N-Z](C₁ to C₆ alkyl)carbonyl(C₁ to C₆ alkyl)amino, halo, halo(C₁ to C₆ alkyl), sulfamoyl, [N-Z](C₁ to C₆ alkyl)sulfonylamino, (C₁ to C₆ alkyl)sulfonylaminocarbonyl, carboxy(C₁ to C₆ alkyl)sulfonyl, carboxy(C₁ to C₆ alkyl)sulfinyl, tetrazolyl, [N-Z]tetrazolylamino, cyano, amidino, amidinothio, SO₃H, formyloxy, formamido, C₃ to C₈ cycloalkyl, (C₁ to C₆ alkyl)sulphamoyl, di(C₁ to C₆ alkyl)sulphamoyl, (C₁ to C₆ alkyl)carbonylaminosulfonyl, 5-oxo-2,5-dihydro[1,2,4]oxadiazolyl, carboxy(C₁ to C₆ alkyl)carbonylamino, tetrazolyl(C₁ to C₆ alkyl)thio, [N-Z]tetrazolyl(C₁ to C₆ alkyl)amino, 5-oxo-2,5-dihydro[1,2,4]thiadiazolyl, 5-oxo-1,2-dihydro[1,2,4]triazolyl, [N-Z](C₁ to C₆ alkyl)amino(C₁ to C₆ alkyl)amino, a group of the formula 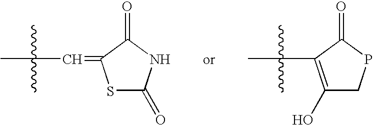
wherein P is O, S or NR¹⁹, or benzyloxycarbonyl(C₁ to C₆ alkyl)thio comprising a phenyl group that is optionally substituted with 1, 2 or 3 groups independently selected from C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano;
Z is H, C₁ to C₆ alkyl, t-butoxycarbonyl, acetyl, benzoyl or benzyl;
R⁴ is selected from an optionally substituted C₁ to C₁₈ hydrocarbyl group wherein up to three C atoms may optionally be replaced by N, O and/or S atoms;
R¹¹, R¹², R¹⁴, R¹⁵, R¹⁷, R¹⁸ and R¹⁹ are independently H or C₁ to C₃ alkyl; and
R¹⁶ is H, C₁ to C₃ alkyl, or acetylamino;
or a pharmaceutically acceptable salt thereof;
with the proviso that when R¹, R³ and R⁵ are all H and R⁴ is methyl, R² may not be methyl.

2. The compound according to claim 1 wherein R¹ and R⁵ are both H.

3. The compound according to claim 1 wherein R² is:
—(CH₂)ₛ—C(R⁶R⁷)ₙ—(CH₂)ₜ-R⁸
wherein:
n is 0 or 1;
s is 0, 1, 2 or 3;
t is 0, 1, 2 or 3; and
R⁸ is selected from H, OH, C₁ to C₁₂ alkyl, (C₁ to C₁₂ alkyl)oxy, C₃ to C₁₂ cycloalkyl, phenyl, benzyloxy, naphthyl, pyridyl, pyrrolyl, imidazolyl, pyrazolyl, pyridazinyl, pyrimidinyl, triazolyl, furanyl, thienyl, furazanyl, oxazolyl, isoxazolyl, thiazolyl, thiazinyl, indolyl, indolinyl, isoindolyl, isoindolinyl, isoquinolinyl, quinolinyl, benzofuranyl, benzothienyl, piperazinyl, piperidinyl, pyrrolidinyl, pyrrolinyl, dihydropyranyl, tetrahydropyranyl, pyranyl, tetrahydrofuranyl, morpholinyl, thiazolidinyl, thiomorpholinyl or thioxanyl (all optionally substituted with 1, 2 or 3 groups independently selected from C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano).

4. The compound according to claim 3, wherein —C(R⁶R⁷)ₙ— is —C(O)—.

5. The compound according to claim 3 wherein s is 1.

6. The compound according to claim 3 wherein t is 0 and R⁸ is a C₃ to C₁₂ cycloalkyl group (optionally substituted with a methyl group) or a branched C₃ to C₁₂ alkyl group.

7. The compound according to claim 3 wherein s is 1, t is 0 and R⁸ is a C₃ to C₁₂ cycloalkyl group or a branched C₃ to C₁₂ alkyl or group.

8. The compound according to claim 3 wherein R⁸ is a t-butyl, cyclohexyl, 1-methylcyclohexyl, 1-methylcyclopentyl or cyclopentyl group.

9. The compound according to claim 3 wherein R⁸ is a t-butyl or cyclopentyl group.

10. The compound according to claim 1 wherein m is 1, R¹¹ is H and R¹² is H

11. The compound according to claim 1 wherein p is 0.

12. The compound according to claim 1 wherein X is C(O)NH.

13. The compound according to claim 1 wherein R⁹ is phenyl substituted with a carboxy, carboxy(C₁ to C₆ alkyl), tetrazolyl, tetrazolyl-N-(C₁ to C₆ alkyl)amino, carboxy(C₁ to C₆ alkyl)thio, (C₁ to C₆ alkyl)oxycarbonyl(C₁ to C₆ alkyl)thio, carboxy(C₁ to C₆ alkyl)sulfonyl, (C₁ to C₆ alkyl)amino, or 5-oxo-2,5-dihydro[1,2,4]oxadiazolyl group, or a benzyloxycarbonyl(C₁ to C₆ alkyl)thio group comprising a phenyl group that is optionally substituted with 1, 2 or 3 groups independently selected from C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano; or R⁹ is a N-[carboxy(C₁ to C₆ alkyl)]indolinyl or N-[carboxy(C₁ to C₆ alkyl)]indolyl group.

14. The compound according to claim 13 wherein the phenyl group is substituted at its 3-position.

15. The compound according to claim 1 wherein R⁴ is
—(CH₂)_{q}-T-R
wherein:
q is 0, 1, 2 or 3;
T is a bond, O, S, NH or N(C₁ to C₆ alkyl); and
R¹⁰ is C₁ to C₁₂ alkyl, C₃ to C₁₂ cycloalkyl, phenyl, naphthyl, pyridyl, pyrrolyl, imidazolyl, pyrazolyl, pyridazinyl, pyrimidinyl, triazolyl, furanyl, thienyl, furazanyl, oxazolyl, isoxazolyl, thiazolyl, thiazinyl, indolyl, indolinyl, isoindolyl, isoindolinyl, isoquinolinyl, quinolinyl, benzofuranyl, benzothienyl, piperazinyl, piperidinyl, pyrrolidinyl, pyrrolinyl, dihydropyranyl, tetrahydropyranyl, pyranyl, tetrahydrofuranyl, morpholinyl, thiazolidinyl, thiomorpholinyl or thioxanyl (all optionally substituted with 1, 2 or 3 groups independently selected from C₁ to C₆ alkyl, (C₁ to C₆ alkyl)oxy, C₃ to C₈ cycloalkyl, (C₃ to C₈ cycloalkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano).

16. The compound according to claim 15 wherein q is 0, T is a bond and R¹⁰ is C₁ to C₁₂ alkyl, C₃ to C₁₂ cycloalkyl, pyridyl or phenyl (all optionally substituted with OMe, NMe₂, CF₃, Me, F, Cl, Br or I).

17. The compound of formula (I) according to claim 16 wherein R⁴ is C₃₋₁₂ cycloalkyl.

18. The compound according to claim 1 wherein R⁴ is cyclohexyl.

19. A compound which is degraded in vivo to yield a compound according to claim 1.

20. A method of treating a gastrin related disorder comprising administering a therapeutically effective amount of a compound of formula (I), according to claim 1, to a patient in need thereof.

21. A method according to claim 20 wherein the gastrin related disorder is a gastrointestinal disorder or cancer.

22. A pharmaceutical composition comprising a compound of formula (I) according to claim 1 together with a pharmaceutically acceptable diluent or carrier.

23-24. (canceled)

25. A method of making a pharmaceutical composition according to claim 22 comprising mixing a compound of formula (I) with a pharmaceutically acceptable diluent or carrier.

26. A pharmaceutical composition comprising a proton pump inhibitor and a compound of formula (I), according to claim 1, together with a pharmaceutically acceptable diluent or carrier.

27. A composition according to claim 26 wherein the proton pump inhibitor is selected from the group consisting of (RS)-rabeprazole, (RS)-omeprazole, lansoprazole, pantoprazole, (R)-omeprazole, (S)-omeprazole, perprazole, (R)-rabeprazole, (S)-rabeprazole, and the alkaline salts thereof.

28. A composition according to claim 26 wherein the proton pump inhibitor and the compound of formula (I) are each in an amount producing a therapeutically beneficial effect in patients suffering from gastrointestinal disorders.

29. A composition according to claim 28 wherein said therapeutically beneficial effect is a synergistic effect on the reduction of acid secretion in patients suffering from gastrointestinal disorders, or the prevention of gastrointestinal disorders in said patients, or the reduction of adverse effects associated with the one of the active ingredients by the other active ingredients.

30. A composition according to claim 26 wherein the amount of each of the active ingredients is equal to or less than that which is approved or indicated in monotherapy with said active ingredient.

31. A kit containing as a first active ingredient a compound of formula (I), according to claim 1, and as a second active ingredient a proton pump inhibitor.

32-37. (canceled)

38. A method of making a pharmaceutical composition, comprising mixing a compound according to claim 1, and a proton pump inhibitor with a pharmaceutically acceptable diluent or carrier.

39. A method of treating a gastrointestinal disorder, comprising the simultaneous or sequential administration of a proton pump inhibitor and a compound according to claim 1 to a subject suffering from said disorder.

40. A method of reducing one or more adverse effects associated with administration of proton pump inhibitors in a patient suffering from a gastrointestinal disorder, comprising administering a compound according to claim 1 to a subject suffering from said disorder.

41. The method according to claim 40, wherein said compound is administered simultaneously or sequentially with said proton pump inhibitor.

42. The method according to claim 40 wherein said adverse effect is hyperplasia.
